(19) United States
(12) Patent Application Publication
(10) Pub. No.: US 2004/0110676 A1
(43) Pub. Date: Jun. 10, 2004

(54) Methods for modulating cell survival

(76) Inventors: Michael Hayden, Vancouver (CA); Abigail Hackam, Baltimore, MD (US); Blair R. Leavitt, Vancouver (CA); Edmond Chan, Vancouver (CA)

Correspondence Address: OPPEDAHL AND LARSON LLP, P O BOX 5068, DILLON, CO, 80435-5068, US

(21) Appl. No.: 10/419,997

(22) Filed: Apr. 22, 2003

Related U.S. Application Data
(63) Continuation-in-part of application No. 10/009,478, filed on May 31, 2002.
Application No. 10/009,478 is a 371 of international application No. PCT/CA01/00495, filed on Apr. 12, 2001.
(60) Provisional application No. 60/374,156, filed on Apr. 22, 2002.

(30) Foreign Application Priority Data
Apr. 13, 2000 (CA) 2,305,088
Dec. 12, 2000 (CA) 2,326,543

(51) Int. Cl. A61K 38/17

(52) U.S. Cl. 514/12; 514/7

(57) ABSTRACT

In general, the invention provides methods, reagents, and uses for modulating cell survival. In various alternative aspects, the invention provides methods of modulating cell survival by administering a huntingtin protein or a biologically-active fragment or variant thereof, or methods of treatment or prophylaxis of a cell degenerative disease or a cell proliferative disease by administering a huntingtin protein or a biologically-active fragment or variant thereof. In various alternative aspects, the invention provides diagnostic assays or methods of assaying test compounds using a huntingtin protein or a biologically-active fragment or variant thereof.

### CROSS-REFERENCE TO RELATED APPLICATIONS

The present application claims the benefit of U.S provisional application 60/374,156, filed Apr. 22, 2002, and is a continuation-in-part of U.S. application Ser. No. 10/009,478, filed May 31, 2002, which is a national stage application of PCT/CA01/00495, filed Apr. 12, 2001, the entire contents of all of which are hereby incorporated by reference.

### FIELD OF THE INVENTION

The invention is, in general, in the field of cell growth regulation. More specifically, the invention provides methods and reagents for modulating cell death and cell proliferation.

### BACKGROUND OF THE INVENTION

Cell growth is intimately associated with cell death, and cells can vary between excess proliferation, normal proliferation, steady state, normal cell senescence and abnormal cell death (FIG. 1). Diseases and disorders often result from a perturbation in the balance between cell growth and death. Thus, cancers may be caused by excessive cell growth, out of proportion to cell death, resulting in rapid proliferation. By contrast, excessive cell death, out of proportion to cell regeneration, can result in the destruction of crucial areas of tissue as observed in degenerative diseases such as Parkinson's Disease, Amyotrophic Lateral Sclerosis, Alzheimer's disease, or Huntington's Disease (HD).

HD is a devastating neurodegenerative disease that usually presents in mid-adult life, affects approximately 1 in 10,000 individuals, and results in psychiatric disturbance, involuntary movement disorder, and cognitive decline associated with inexorable progression to death, typically 17 years following onset. The lesion in HD has been traced to the expansion of a CAG trinucleotide repeat in the first exon of the HD gene, IT15, located on chromosome 4 (4p 16.3) (Huntington Disease Collaborative Research Group, 1993). Alleles containing expansions of greater than 35 CAG repeats are associated with the clinical phenotype of HD, with an earlier age of onset occurring with higher CAG repeat sizes (Andrew et al. (1993) Nat. Genet. 4, 398-403).

The mutation in the HD gene results in a protein, huntingtin (htt), with an expanded polyglutamine tract. Htt is a large protein of uncertain function that is ubiquitously expressed in many tissues of the body, but which has the highest levels in brain and testis (Sharp and Ross, (1996) Neurobiol. Dis. 3, 3-15). Proteolytic cleavage of htt, possibly by caspases, produces N-terminal htt fragments containing the expanded polyglutamine tract (Goldberg et al. (1996) Nat. Genet. 13, 442-449; Wellington et al. (1998) J. Biol. Chem. 273, 9158-9167; Wellington et al. (2000) J. Biol. Chem. 275, 19831-19838). N-terminal fragments of mutant expanded htt have altered cellular interactions (Li et al. (1995) Nat. Genet. 25, 385-389; Burke et al. (1996) Nat. Med. 2, 347-349; Bao et al. (1996) Proc. Nat. Acad. Sci. USA 93, 5037-5042; Wanker et al. (1996) Hum. Mol. Genet. 6,487495; Kalchman et al. (1996) J. Biol. Chem. 271, 19385-19394; Kalchman et al. (1997) Nat. Genet. 16, 44-53), nuclear localisation (Davies et al. (1997) Cell 90, 537-548; DiFiglia et al. (1997) Science 277, 1990-1993; Becher et al. (1998) Neurobiol. Dis. 4, 387-397; Hackam et al. (1998) Cell. Biol. 141, 1097-1105; Schilling et al. (1999) Hum. Mol. Genet. 8, 397-407; Hodgson et al. (1999) Neuron 23, 181-192; Gutekunst et al. (1999) J. Neurosci. 19, 2522-2534; Wheeler et al. (2000) Hum. Mol. Genet. 9, 503-513; Li et al. (2000) Nat. Genet. 25, 385-389), and are directly toxic to neuronal cells in a variety of in vitro model systems (Martindale et al. (1998) Nat. Genet. 18, 150-154; Sandou et al. (1998) Cell 95, 55-66; Hackam et al. (1998) Cell. Biol. 141, 1097-1105). These htt fragments are also prone to intracellular aggregation and inclusion formation (Hackam et al. (1998) Cell. Biol; 141, 1097-1105; Martindale et al. (1998) Nat. Genet. 18, 150-154; Wang et al. (1999) Neuroreport. 10, 2435-2438, Cooper et al. (1998) Hum. Mol Genet. 7, 783-7 90), although the relevance of htt aggregation to the pathogenesis of HD remains unclear (reviewed in Sisoda (1998) Cell 95,14). The expansion of polyglutamine residues in htt has been proposed to result in a novel toxic gain of function of the mutant protein (MacDonald and Gusella (1996) Curr. Opin. Neurobiol. 6, 638-643). Htt has also been implicated in haematopoiesis (Metzler et al. (2000) Hum Mol Genet 9, 387-94), and a lower incidence of cancer among patients with HD, which appears to be related to intrinsic biologic factors, has been observed (Sorensen et al. (1999) Cancer 86, 1342-1346).

Mice homozygous for targeted disruption of Hdh (−/−), the murine homologue of the HD gene, die at embryonic day 7.5 (Nasir et al. (1995) Cell 81, 811-823; Duyao et al. (1995) Science 269, 407-410; Zeitlin et al. (1995) Nat. Genet. 11, 155-162). Mice with decreased levels of htt following targeted insertion of a neo construct into the Hdh gene have aberrant brain development and perinatal lethality (White et al. (1997) Nat. Genet. 17, 404-410). Mice heterozygous for targeted disruption of the Hdh gene (+/−) express half the normal levels of endogenous htt and develop neuronal degeneration in the basal ganglia in adulthood (O'Kusky et al. (1999) Brain. Res. 818, 468-479).

Mice containing yeast artificial chromosome (YAC) transgenic for the entire genomic region of the human HD gene, including all its regulatory sequences (Hodgson et al. (1999) Neuron 23, 181-192), have been generated to study the phenotypic effects of varying endogenous htt levels on mice expressing transgenic htt. The appropriate expression of human htt during development in YAC transgenic mice was demonstrated by the ability of the human transgene to rescue the embryonic lethality of Hdh nullizygous mice (−/−) (Hodgson et al. (1996) Hum. Mol. Genet. 5, 1875-1885). In addition, YAC transgenic mice that express human htt with 18 polyglutamines (YAC 18) corresponding to a CAG repeat length observed in unaffected persons, 46 polyglutamines (YAC46) corresponding to a CAG repeat length observed in adult-onset HD patients, and 72 polyglutamines (YAC72) corresponding to a repeat length causing juvenile-onset HD (Hodgson et al. (1999) Neuron 23, 181-192) express similar levels of transgenic human htt differing only in polyglutamine expansion length. YAC18 mice have no observable phenotype up to 24 months of age, indicating that human htt with a polyglutamine tract of normal length is not pathogenic in mice. However, mice transgenic for mutant htt with an expanded polyglutamine develop a progressive phenotype characterized by behavioral, cellular and neuropathologic abnormalities similar to those observed in HD (Hodgson et al. (1999) Neuron 23, 181-192).

Given the devastating effects of conditions associated with aberrant cell growth regulation, in diseases and disorders exemplified by cancer or HD, it would be useful to have reagents and methods for promoting or inhibiting cell survival.

### SUMMARY OF THE INVENTION

In various alternative aspects, the invention provides methods and reagents for modulating cell survival. In one aspect, the invention provides a method of modulating cell survival in a subject in need of such modulation, by administering an effective amount of a huntingtin protein or a biologically-active fragment or variant thereof. In an alternative aspect, the invention provides a method of treatment or prophylaxis of a cell degenerative disease by administering a huntingtin protein or a biologically-active fragment or variant thereof, to a subject in need of such treatment. In another alternative aspect, the invention provides a method of treatment or prophylaxis of a cell proliferation disease by administering a huntingtin protein or a biologically-active fragment or variant thereof, to a subject in need of such treatment. The subject may be a human. In some embodiments, the invention provides for the modulation of cell death or apoptosis, or of cell proliferation.

In alternative embodiments, the method may include administering a nucleic acid molecule encoding the huntingtin protein or biologically-active fragment may be administered, for example, as a gene therapy; administering a nucleic acid molecule complementary to a nucleic acid encoding the huntingtin protein or biologically-active fragment; or adminstering an antibody that specifically binds the huntingtin protein or biologically-active fragment.

In alternative embodiments, the cell degenerative disease may be a neurodegenerative disease, Huntington's disease, Alzheimer's disease, Parkinson's disease, amyotrophic lateral sclerosis, multiple sclerosis, polyglutamine diseases, spinocerebellar ataxias, autosomal dominant cerebellar ataxia with retinal degeneration, spinobulbar muscular atrophy (SBMA), dentatorubralpallidoluysian atrophy (DRPLA), Machado-Joseph disease, stroke, epilepsy, spinal cord injury, physical trauma, or retinal degeneration.

In alternative embodiments, the cell proliferative disease may be cancer, testicular cancers, embryonic cancers, leukemias, haematopoietic diseases, psoriasis, atherosclerosis, inflammatory diseases, or dermatological diseases.

In alternative aspects, the invention provides a method of assaying a test compound, by providing a system including a huntingtin protein or biologically-active fragment, contacting the system with the test compound, and determining whether the test compound modulates the phosphorylation of the huntingtin protein or fragment. In alternative embodiments, the assaying is done in vitro and further includes providing a kinase, for example AKT, capable of phosphorylating the huntingtin protein or biologically-active fragment.

In alternative aspects, the invention provides a method of assaying a test compound, by providing a system including a huntingtin protein or biologically-active fragment, contacting the system with the test compound, and determining whether the test compound modulates a function of the huntingtin protein or fragment, where the function may be apoptosis inhibition, aggregation inhibition, or cell proliferation.

In alternative aspects, the invention provides a method of ameliorating the cytotoxic effects of a therapeutic compound, for example, a pro-apoptotic compound such as tamoxifen, by co-administering a huntingtin protein or a biologically-active fragment or variant with the therapeutic compound.

In alternative aspects, the invention provides a method for diagnosing cell proliferation, by determining the level of a huntingtin protein or nucleic acid molecule in a test sample, for example, from gastric cancer or breast cancer tissue, and in a control sample, where the test sample is positive for cell proliferation if the level of huntingtin is higher in the test sample when compared to the level of huntingtin in the control sample.

In alternative embodiments of various aspects of the invention, the variant may be an agonist of a huntingtin protein or an antagonist of a huntingtin protein. In alternative embodiments, the huntingtin protein may be full-length huntingtin protein; wild-type huntingtin protein; mutant huntingtin protein; phosphorylated huntingtin protein, for example, phosphorylated on serine 421, threonine 1024, or threonine 2068 of the human huntingtin protein (SEQ ID NO: 1); unphosphorylated huntingtin protein; constitutively phosphorylated huntingtin protein. In alternative embodiments, the biologically-active fragment may be a C-terminal fragment of a huntingtin protein, for example, amino acids 585-3144 of the human huntingtin protein (SEQ ID NO: 1), or about the C-terminal half of the human huntingtin protein (SEQ ID NO: 1).

“Cell survival” refers to a reduction or decrease in cell death, or a promotion or increase in cell proliferation. “Cell death” or “apoptosis,” defines a specific execution of programmed cell death that can be triggered by several factors (Krarnmer et al. (1991) “Apoptosis in the APO-1 System”, Apoptosis: The Molecular Basis of Cell Death, pp. 87-99 Cold Spring Harbor Laboratory Press). One of the factors triggering apoptosis is loss of cell anchorage (Meredith, J. E., Jr., B. Fazeli, et al. (1993). “The extracellular matrix as a cell survival factor.” Molecular Biology of the Cell. 4(9): 953-61), a phenomenon known as “anoikis” (Frisch, S. M. and H. Francis (1994). “Disruption of epithelial cell-matrix interactions induces apoptosis.” Journal of Cell Biology. 124(4): 619-26). “Cell proliferation” refers to excessive or aberrant cell growth. When the normal function of cell survival go awry, the cause or the result can be cell degenerative or cell proliferative diseases, including cancer, viral infections, autoimmune disease/allergies, cardiovascular diseases, neurodegeneration, etc.

“Modulating” or “modulates” means changing, by either increase or decrease.

A “huntingtin” or “htt” protein or polypeptide is a protein, the mutant form of which has been shown to be associated with Huntington's disease (HD). While the invention encompasses htt from various species, such as mouse, rat, Drosophila, C. elegans, etc., an exemplary htt protein from humans has an amino acid sequence (SEQ ID NO: 1), listed on GenBank as Accession Number NP_{—}002102, and is encoded by a nucleic acid sequence (SEQ ID NO: 2), listed on GenBank as Accession Number NM_{—}002111. The “C terminus” of htt indicates, generally, the half of the wild-type or mutant htt protein that includes the C-terminus, and possesses biological activity as described herein. The term is used synonymously with C-terminal fragment or C-terminal domain. C-terminal fragments of htt also include amino acids 585-3144 of SEQ ID NO: 1, and any biologically-active fragments thereof. The “N terminus” or “N-terminal fragment” indicates, generally, the half of the wild-type htt protein that includes the N-terminus and any biologically-active fragments thereof. A “full-length” htt protein has the entire amino acid sequence of a htt protein that is naturally found in an organism of the species that normally expresses that htt protein. For example, a full length human htt protein may have the amino acid sequence of SEQ ID NO: 1. A “wild type” htt protein possesses, generally, a polyglutamine tract of less than 35 glutamines, and is not associated with the development of HD. A “mutant” htt protein possesses, generally, a polyglutamine tract of more than 35 glutamines, and is associated with the development of HD of varying severity, depending on the length of the polyglutamine tract. A htt protein according to the invention may have a sequence substantially identical to that of SEQ ID NO: 1.

A “phosphorylated” htt protein is post-translationally modified on any amino acid residue capable of being phosphorylated in vivo. Phosphorylated htt proteins may be phosphorylated, for example, on serine 421, threonine 1024, or threonine 2068. An “unphosphorylated” htt protein may be incapable of being phosphorylated on an amino acid residue capable of being phosphorylated in vivo, for example, by mutation of that residue to an amino acid that is not capable of being phosphorylated. A mutation of a serine to an alanine in a polypeptide sequence, for example, results in a protein that is not capable of being phosphorylated at that particular position in the polypeptide sequence. A htt protein that possesses an alanine at position 421 of SEQ ID NO: 1 instead of a serine is such an “unphosphorylated” htt protein. An unphosphorylated htt protein may also be a protein that is capable of being phosphorylated in vivo, but is not phosphorylated due to, for example, the presence of an inhibitor, for example, a kinase inhibitor; due to an antibody that interferes with the phosphorylation site; or due to the activity of a phosphatase. A “constitutively phosphorylated” htt protein is a protein that possesses a mutation at an amino acid residue that is capable of being phosphorylated in vivo, where the mutation mimics phosphorylation at that residue, and the resultant polypeptide possesses the biological activity of a phosphorylated polypeptide. Generally, mutation of a phosphorylatable reside to a glutamic acid or aspartic acid residue results in constitutive phosphorylation.

A “biologically-active fragment” of a htt protein includes an amino acid sequence found in a naturally-occurring htt protein that is capable of modulating apoptosis or cell death, cell proliferation, or protein aggregation, for example, aggregation caused by the expansion of a polyglutamine tract, as described herein or known to those of ordinary skill in the art. A “variant” of a htt protein includes a modification, for example, by deletion, addition, or substitution, of an amino acid sequence found in a naturally-occurring htt protein that is capable of modulating apoptosis or cell death, cell proliferation, or protein aggregation, as described herein or known to those of ordinary skill in the art.

A “protein,” “peptide” or “polypeptide” is any chain of two or more amino acids, including naturally occurring or non-naturally occurring amino acids or amino acid analogues, regardless of post-translational modification (e.g., glycosylation or phosphorylation). An “amino acid sequence”, “polypeptide”, “peptide” or “protein” of the invention may include peptides or proteins that have abnormal linkages, cross links and end caps, non-peptidyl bonds or alternative modifying groups. Such modified peptides are also within the scope of the invention. The term “modifying group” is intended to include structures that are directly attached to the peptidic structure (e.g., by covalent coupling), as well as those that are indirectly attached to the peptidic structure (e.g., by a stable non-covalent association or by covalent coupling to additional amino acid residues, or mimetics, analogues or derivatives thereof, which may flank the core peptidic structure). For example, the modifying group can be coupled to the amino-terminus or carboxy-terminus of a peptidic structure, or to a peptidic or peptidomimetic region flanking the core domain. Alternatively, the modifying group can be coupled to a side chain of at least one amino acid residue of a peptidic structure, or to a peptidic or peptido- mimetic region flanking the core domain (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the peptidic structure can be attached by means and using methods well known in the art for linking chemical structures, including, for example, amide, alkylamino, carbamate or urea bonds.

A “substantially identical” sequence is an amino acid or nucleotide sequence that differs from a reference sequence only by one or more conservative substitutions, as discussed herein, or by one or more non-conservative substitutions, deletion, or insertions located at positions of the sequence that do not destroy htt biological function as described herein. Such a sequence can be at least 60% or 75%, or more generally at least 80%, 85%, 90%, or 95%, or as much as 99% identical at the amino acid or nucleotide level to the sequence used for comparison. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine). Examples of useful software include the programs, Pile-up and PrettyBox. Such software matches similar sequences by assigning degrees of homology to various substitutions, deletions, substitutions, and other modifications.

An “agonist” is a compound, including a peptide or peptide analogue, that possesses the biological activities of a naturally-occurring htt protein, and triggers biochemical responses that would be triggered by a naturally-occurring htt protein. Thus an agonist, according to the invention, may modulate cell survival by, for example, inhibiting cell death or apoptosis, or promoting cell proliferation. An agonist compound will have biological activity if it competes with a htt protein, peptide or peptide analogue, as described herein, in its ability to inhibit an apoptotic or aggregation response, or promote a proliferative response when compared to a htt protein, peptide or peptide analogue. Generally, an agonist compound will exhibit at least 20% modulation, or at least 30% to 50% modulation, or even over 80% or over 100% modulation when compared to a htt protein, peptide or peptide analogue.

An “antagonist” is a compound, including a peptide or peptide analogue, that nullifies the biological activities of a naturally-occurring htt protein, and is incapable of triggering the biochemical responses that would be elicited by a naturally-occurring htt protein. Thus an antagonist, according to the invention, may modulate cell survival by, for example, promoting cell death or apoptosis, or inhibiting cell proliferation. An antagonist compound will have biological activity if it competes with a htt protein, peptide or peptide analogue, as described herein, in its ability to modulate a apoptotic, aggregation, or proliferative response when compared to a htt protein, peptide or peptide analogue. Generally, an antagonist compound will exhibit at least 20% modulation, or at least 30% to 50% modulation, or even over 80% or over 100% modulation when compared to a htt protein, peptide or peptide analogue. Antisense oligonucleotide molecules, or antibodies that interfere with htt function, may be antagonists. An antagonist may also be a biologically inactive form, or fragment, of htt protein that interferes with the action of the wild-type protein, such as a dominant negative mutant of a htt protein.

A “nucleic acid molecule” is any chain of two or more nucleotides including naturally occurring or non-naturally occurring nucleotides or nucleotide analogues. A nucleic acid molecule is “complementary” to another nucleic acid molecule if it hybridizes, under conditions of high stringency, with the second nucleic acid molecule.

An antibody “specifically binds” an antigen when it recognises and binds the antigen, but does not substantially recognise and bind other molecules in a sample, having for example an affinity for the antigen which is 10, 100, 1000 or 10000 times greater than the affinity of the antibody for another reference molecule in a sample.

A “cell degenerative disease” is a disorder or condition characterised by the progressive or acute loss of cells or tissue. A progressive cell degenerative disease could result from, for example, a hereditary deficiency or environmental factors leading to a gradual loss of cells or tissue, often over a period of years. An acute cell degenerative disease could result from trauma, such as physical injury or stroke.

A “cell proliferative disease” is a disorder or condition characterised by excessive or aberrant cell growth.

A “test compound” is any chemical compound, be it naturally-occurring or artificially-derived. Test compounds may include, without limitation, peptides, polypeptides, synthesised organic molecules, naturally occurring organic molecules, and nucleic acid molecules. A test compound can “compete” with a known compound such as a htt protein or fragment thereof by, for example, interfering with modulation of apoptosis or cell death, cell proliferation, or protein aggregation by htt or the known compound, or by interfering with any biological response induced by htt or the known compound. Generally, a test compound will exhibit at least 20% modulation, or at least 30% to 50% modulation, or even over 80% or over 100% modulation when compared to a htt protein, peptide or peptide analogue.

By “contacting” is meant to submit an animal, cell, lysate, extract, or molecule derived from a cell to a test compound.

By “determining” is meant analyzing the effect of a test compound on the test system. The means for analyzing may include, without limitation, antibody labeling, cell proliferation assays, immunoprecipitation, in vivo and in vitro phosphorylation assays, cell death assays, anoikis assays, ultrastructural analysis, histological analysis, or any other methods known to those skilled in the art.

Other features and advantages of the invention will be apparent from the following description of the drawings and the invention, and from the claims.

### BRIEF DESCRIPTION OF THE DRAWINGS

FIG. 1 presents a schematic representation of the balance between normal cell proliferation and cell death.

FIGS. 2A-I are semi-thin sections showing the testicular morphology of yeast artificial chromosome (YAC) rescued htt knockout mice. The YACs used to rescue mice contain the htt gene with 18, 46 or 72 CAG repeats. Semi-thin sections of testes reveal the gross testicular morphology of mice with varying amounts of endogenous htt rescued with YAC18 (FIGS. 2A-C), YAC46 (FIGS. 2D-F), and YAC72 (FIGS. 2G-I) transgenes.

FIG. 3 shows electron micrographs of protein aggregates in YAC72 rescued htt knockout mice.

FIG. 4 shows electron micrographs of degenerating testicular cells from YAC72 rescued htt knockout mice.

FIG. 5 is a bar graph showing that the C-terminus of htt protein is anti-apoptotic.

FIG. 6 is a bar graph showing that the C-terminus of htt protein confers protection against htt toxicity in NT2 cells.

FIG. 7 is a bar graph showing that the C-terminus of htt protein rescues HIP- 1 toxicity in NT2 cells.

FIGS. 8A-B are bar graphs showing that the C-terminus of htt protein reduces mutant htt protein aggregate formation.

FIGS. 9A-J show results of studies with YAC transgenic mice expressing increased levels of wild-type human htt. FIG. 9A is a bar graph quantifying degenerating hippocampal neurons following kainic acid-induced seizures in mice expressing 2-3 times the endogenous levels of wild-type htt (212 line) and littermate controls (FVB/NJ). Average numbers of degenerating neurons per animal identified by Fluoro-Jade™ labeling are expressed for the CA1, CA3 and total hippocampal regions. Data are expressed as mean +/− SEM with significance determined using a two-tailed Students t-test. FIGS. 9B-J show hippocampal sections of degenerating neurons (arrows) using Fluoro-Jade™ labeling (FIG. 9B shows line 212 at 20×, FIG. 9C shows FVB/NJ at 20×, FIG. 9D shows FVB/NJ at 100×), silver staining (FIG. 9E shows line 212 at 20×, FIG. 9F shows FVB/NJ at 20×, FIG. 9G shows FVB/NJ at 100×), and TUNEL staining (FIG. 9H shows line 212 at 20×, FIG. 9I shows FVB/NJ at 20× and FIG. 9J shows FVB/NJ at 100×) within the hippocampus following KA-induced seizures.

FIG. 10 is a bar graph quantifying hippocampal and cerebellar DEVD-ase activity following kainic acid-induced seizures in mice expressing 2-3 times the endogenous levels of wild-type htt (212 line) and littermate controls (FVB/NJ). YAC transgenic mice expressing increased levels of wild-type human htt have decreased caspase-3 activation following kainic acid-induced seizures. Data are expressed as mean +/− SEM with significance determined using a two-tailed Students t-test.

FIGS. 11A-E show results of the rescue of the Hdh nullizygous lethal phenotype by YAC transgenes expressing mutant htt. FIG. 11A shows the resultant genotypes for the F2 offspring of a cross between a YAC72 transgene positive, Hdh gene heterozygous mouse (+,+/− genotype) and a transgene negative, Hdh heterozygous mouse (−,+/− genotype). The upper PCR bands represent the presence or absence of the YAC transgene and the lower bands represent the state of the endogenous Hdh gene. The mouse represented in lane two has the YAC72 transgene, but lacks the endogenous Hdh gene (+,−/− genotype). FIG. 11B shows the expected 1:2:1 ratio of genotypes for all of the YAC transgenes examined in the F2 offspring of the experimental breedings. FIG. 11C is a western blot analysis of htt protein expression confirming the absence of endogenous htt protein in Hdh nullizygous mice (−/−) compared to wild-type (+/+) and demonstrating similar levels of human transgenic htt expression in YAC18, YAC46, and YAC72 rescued Hdh nullizygous mice (+,−/−). Average testicular weight and epididymal sperm counts for YAC72+/+, YAC72+/−, and YAC72−/− mice at four months of age are shown in FIG. 11D and FIG. 11E, respectively. YAC72−/− mice had significant testicular atrophy (p<10⁻⁵) and decreased sperm counts (<10⁻⁵) compared to YAC72+/+ and YAC72+/− mice.

FIGS. 12A-I show testicular morphology of YAC transgene rescued Hdh nullizygous mice. Semi-thin sections of testes stained with toluidine blue from 8-month-old mice reveal the gross testicular morphology of mice with the YAC 18 (FIGS. 12A-C), YAC46 (FIGS. 12D-F), and YAC72 (FIGS. 12G-I) htt transgenes and either 100% of endogenous htt levels (+/+), 50% of endogenous htt levels (+/−) or absence of endogenous htt (−/−). Massive degeneration of spermatogenic cells occurs in the seminiferous tubules of mice expressing mutant htt with 46 or 72 polyglutamine repeats (FIGS. 12I and 12F). The cell death is most pronounced in YAC72 (FIG. 12I), intermediate in YAC46 (FIG. 12F), and not present in YAC18 (FIG. 12C) rescued Hdh nullizygous mice. The human HD transgene in each of these lines of mice is identical except for the length of the CAG repeat, and these results suggest that this novel cell death phenotype is CAG repeat length-dependent. Increasing levels of endogenous htt markedly reduced the amount of spermatogenic cell degeneration (FIGS. 12D, 12E, 12G, and 12H) observed in YAC46 and YAC72 mice. (Bar=100 μm.)

FIGS. 13A-G show morphologic, biochemical, and ultrastructural evidence for apoptotic cell death in the testes of YAC72 mice lacking endogenous htt. Massive death of spermatogenic cells was observed in YAC72 mice lacking endogenous htt by toluidine blue staining (FIG. 13A) which revealed decreased numbers of spermatogenic cells, and a disordered epithelium filled with vacuoles compared to the well large numbers of spermatocytes in well ordered stratified epithelium of YAC72+/+ mice (FIG. 13C). Increased apoptosis was evident in the testes of YAC72−/− mice by increased TUNEL™ labeling of spermatogenic cells (arrows in FIG. 13B) compared to YAC72 mice with normal levels of endogenous htt (FIG. 13D). Electron microscopic analysis of degenerating testicular cells from YAC72 (−/−) mice also provided evidence of apoptosis. Ultrastructural analysis of testes from YAC72 mice nullizygous for Hdh reveals massive cell death of spermatids, phagocytosis of degenerating cells, and formation of multinucleated giant cells. The epithelium of YAC72 mice tacking endogenous htt was characterized by degenerating spermatids filled with cytoplasmic vacuoles (FIG. 13E), phagosomes containing shrunken electron-dense spermatids engulfed within Sertoli cells (FIG. 13F), and spermatogenic giant cells (FIG. 13G). Bars (FIGS. 13A-D)=100 μm, (FIG. 13E)=10 μm, (FIG. 13F)=5 μm, (FIG. 13G) 10 μm.

FIGS. 14A-E show protein aggregates in YAC72 (−/−) mice. Ultrastructural analysis of the testes of YAC72 mice lacking endogenous htt revealed the occasional presence of abnormal aggregates of intracellular protein (arrows) within elongate spermatids (FIG. 14A), sertoli cells (FIG. 14B), and sperm tails (FIG. 14C). The composition of these protein aggregates is not entirely clear, but they resemble the ultrastructural appearance of htt aggregates found in human HD brain tissue. Ectopic microtubule bundles (FIG. 14D) and manchettes (FIG. 14E) were also identified (asterisks). The bundle in FIG. 14E is in a spermatogonium. N-nucleus. Bars (FIGS. 14A-B)=5 μm, (FIGS. 14C-D)=1 μm, (FIG. 14E)=5 μm.

FIGS. 15A-J show immunocytochemical analysis of protein aggregates and actin distribution in sections from the testes of YAC72−/− mice. Abnormal protein aggregates within degenerating spermatogenic cells in the testes of YAC72−/− mice contain htt (FIG. 15A, phase, and FIG. 15B, immunofluorescence). In normal epithelium (FIG. 15E, phase, and FIG. 15F, fluorescence), actin filaments in Sertoli cells are concentrated in unique adhesion plaques (ectoplasmic specializations) that occur at apical sites of attachment to spermatids (Apical) and at basal sites of attachment to neighbouring Sertoli cells (Basal). In YAC72−/− mice (FIGS. 15G-J), filament bundles (asterisks) in apical regions occur in areas not associated with spermatid heads, although filament bundles at basal sites occur in their normal position. Bars (FIGS. 15A-D)=10 μm, (FIGS. 15E-J)=50 μm.

FIG. 16 is a bar graph showing the effect of htt over-expression on body weight.

FIG. 17 is a bar graph showing the effect of transfecting NIH3T3 cells with a wild-type htt gene or a known oncogene (ras).

FIG. 18 is an autoradiograph showing phosphorylation of serine 421 of htt by AKT in vitro.

FIG. 19 is a schematic diagram of the strategy used to construct the S421 mutants.

FIG. 20 shows photographs depicting stable retroviral transfection of NIH3T3 cells.

FIG. 21 is a bar graph showing soft-agar assay results with retrovirally transfected NIH3T3 cells.

FIG. 22 is a bar graph showing htt expression in gastric cancer.

FIG. 23 is a bar graph showing htt expression in breast cancer.

FIG. 24 is a bar graph showing the effect of htt on NIH/3T3 cells in an anoikis assay.

FIGS. 25A-B are bar graphs showing the effect of phosphorylation of htt on HBL-100 cells in an anoikis assay.

FIGS. 26A-B show the effect of phosphorylation of htt on HBL-100 cells in an anoikis assay.

FIG. 27 is an amino acid sequence of human huntingtin (GenBank Accession No. NP_{—}002102) (SEQ ID NO: 1).

FIGS. 28A-C is a nucleic acid sequence of human huntingtin (GenBank Accession No. NM_{—}002111) (SEQ ID NO: 2)

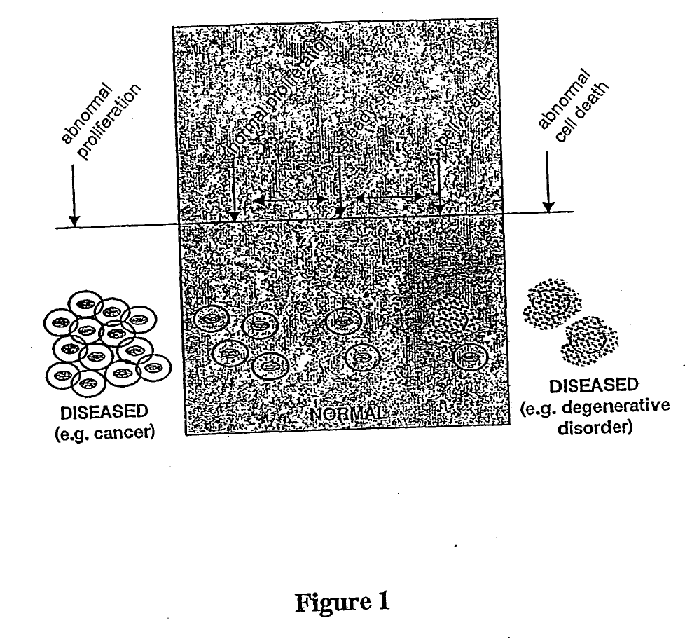

### DETAILED DESCRIPTION OF THE INVENTION

In general, the invention provides methods and uses for htt in modulating cell survival. It has been found that htt is capable of inhibiting or decreasing cell death or apoptosis and promoting cell proliferation. It has also been found that phosphorylation of htt plays a role in its function, and that dephosphorylated or unphosphorylatable htt is capable of promoting or increasing cell death or apoptosis, and inhibiting or decreasing cell proliferation. Conversely, phosphorylated htt inhibits apoptosis and promotes cell proliferation. Phosphorylation also decreases the cellular toxicity of htt. Various alternative embodiments of the invention are described below. These embodiments include, without limitation, use of htt to modulate cell survival, to diagnose cell proliferation, or to assay test compounds.

Use of Htt to Modulate Cell Survival

The methods of the invention may be used to promote cell survival, for example, by inhibiting cell death or apoptosis, or by promoting cell proliferation. The methods of the invention may also be used to inhibit cell survival by promoting cell death or apoptosis, or by inhibiting cell proliferation. Since htt is ubiquitously expressed, the methods and reagents according to the invention may be used in a wide range of cells types to modulate cell survival in a variety of cell proliferative or cell degenerative disorders. The subject or patient to be treated may be a patient that is not suffering from HD, or a patient having an IT15 allele that is not associated with a HD disease state (such as an IT15 allele having 35 or fewer CAG repeats in the first exon of the HD gene). Htt can be used to treat an “apoptotic disease” state, defined for example as a condition characterized.by the occurrence of undesirably high levels of apoptosis, for example certain neurodegenerative diseases such as Alzheimer's disease, Parkinson's disease, amyotrophic lateral sclerosis, multiple sclerosis, restenosis, stroke, and ischemic brain injury and other diseases in which neuronal cells undergo undesired apoptotic cell death.

Wild-type htt proteins, phosphorylated htt proteins, or fragments or agonists thereof, or antagonists of dephosphorylated or unphosphorylatable htt proteins, or fragments thereof, may be used to promote cell survival and, for example, treat diseases or disorders that result in inappropriate cell death, including chronic or progressive cell death, or acute cell death. Such diseases or disorders may include, without limitation, neurodegenerative diseases, such as HD, Alzheimer's disease, Parkinson's disease, amyotrophic lateral sclerosis, multiple sclerosis; polyglutamine diseases, such as HD, spinocerebellar ataxias, autosomal dominant cerebellar ataxia with retinal degeneration, spinobulbar muscular atrophy (SBMA), or dentatorubralpallidoluysian atrophy (DRPLA), or Machado-Joseph disease; stroke; epilepsy, spinal cord injury; physical trauma; or retinal degeneration.

Dephosphorylated or unphosphorylatable htt proteins, or fragments or agonists thereof, or antagonists of wild-type htt proteins, phosphorylated htt proteins, or fragments thereof, may be used to inhibit cell survival and, for example, treat diseases or disorders that result in inappropriate cell proliferation. Such diseases or disorders may include, without limitation, cancer, such as testicular cancers, embryonic cancers, leukemias; haematopoietic diseases; psoriasis, atherosclerosis; dermatological diseases, such as pemphigus vulgaris and pemphigus foleaceus; or inflammatory disorders.

Compounds

In one aspect, compounds according to the invention include non-pathogenic htt polypeptides, for example, the wild-type human htt protein (SEQ ID NO:1), as well as homologs, such as the mouse, rat, or zebrafish homologs, and fragments thereof. A skilled person will recognise that non-pathogenic htt polypeptides include those proteins that contain less than about 35 consecutive glutamines, and thus may differ from the protein of SEQ ID NO:1 in the length of the polyglutamine tract. Compounds within the scope of the invention include htt polypeptides that are phosphorylated or unphosphorylated, including polypeptides that are constitutively phosphorylated, or that are unphosphorylatable. Compounds within the scope of the invention also include fragments of htt polypeptides, for example, C-terminal polypeptides (e.g., a fragment that corresponds to amino acids 585-3144 of SEQ ID NO: 1, or that corresponds to about the C-terminal half of SEQ ID NO: 1). N-terminal fragments, for example, fragments corresponding to about the N-terminal half of SEQ ID NO: 1 are also within the scope of the invention.

In alternative embodiments, a compound according to the invention can be a non-peptide molecule as well as a peptide or peptide analogue. A non-peptide molecule can be any molecule that exhibits biological activity as described herein. Biological activity can, for example, be measured in terms of ability to elicit a apoptosis, aggregation, or cell-proliferation response. Compounds capable of increasing wild-type htt expression levels are also within the scope of the invention.

Compounds can be prepared by, for example, replacing, deleting, or inserting an amino acid residue of a htt protein, peptide or peptide analogue, as described herein, with other conservative amino acid residues, i.e., residues having similar physical, biological, or chemical properties, and screening for biological function.

It is well known in the art that some modifications and changes can be made in the structure of a polypeptide without substantially altering the biological function of that peptide, to obtain a biologically equivalent polypeptide. In one aspect of the invention, polypeptides of the present invention also extend to biologically equivalent peptides that differ from a portion of the sequence of the polypeptides of the present invention by conservative amino acid substitutions. As used herein, the term “conserved amino acid substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the finction of the peptide by routine testing.

As used herein, the term “amino acids” means those L-amino acids commonly found in naturally occurring proteins, D-amino acids and such amino acids when they have been modified. Accordingly, amino acids of the invention may include, for example: 2-Aminoadipic acid; 3-Aminoadipic acid; beta-Alanine; beta-Aminopropionic acid; 2-Aminobutyric acid; 4-Aminobutyric acid; piperidinic acid; 6-Aminocaproic acid; 2-Aminoheptanoic acid; 2-Aminoisobutyric acid; 3-Aminoisobutyric acid; 2-Aminopimelic acid; 2,4 Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; sarcosine; N-Methylisoleucine; 6-N-methyllysine; N-Methylvaline; Norvaline; Norleucine; and Ornithine.

In some embodiments, conserved amino acid substitutions may be made where an amino acid residue is substituted for another having a similar hydrophilicity value (e.g., within a value of plus or minus 2.0), where the following may be an amino acid having a hydropathic index of about −1.6 such as Tyr (−1.3) or Pro (−1.6)s are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (0); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In alternative embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gln (−3.5); Asp (−3.5); Asn (−3.5); Lys (−3.9); and Arg (−4.5).

In alternative embodiments, conserved amino acid substitutions may be made where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gln, Tyr.

Conservative amino acid changes can include the substitution of an L-amino acid by the corresponding D-amino acid, by a conservative D-amino acid, or by a naturally-occurring, non-genetically encoded form of amino acid, as well as a conservative substitution of an L-amino acid. Naturally-occurring non-genetically encoded amino acids include beta-alanine, 3-amino-propionic acid, 2,3-diamino propionic acid, alpha-aminoisobutyric acid, 4-amino-butyric acid, N-methylglycine (sarcosine), hydroxyproline, ornithine, citrulline, t-butylalanine, t-butylglycine, N-methylisoleucine, phenylglycine, cyclohexylalanine, norleucine, norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, homoarginine, N-acetyl lysine, 2-amino butyric acid, 2-amino butyric acid, 2,4,-diamino butyric acid, p-aminophenylalanine, N-methylvaline, homocysteine, homoserine, cysteic acid, epsilon-amino hexanoic acid, delta-amino valeric acid, or 2,3-diaminobutyric acid.

In alternative embodiments, conservative amino acid changes include changes based on considerations of hydrophilicity or hydrophobicity, size or volume, or charge. Amino acids can be generally characterized as hydrophobic or hydrophilic, depending primarily on the properties of the amino acid side chain. A hydrophobic amino acid exhibits a hydrophobicity of greater than zero, and a hydrophilic amino acid exhibits a hydrophilicity of less than zero, based on the normalized consensus hydrophobicity scale of Eisenberg et al. (J. Mol. Bio. 179:125-142, 184). Genetically encoded hydrophobic amino acids include Gly, Ala, Phe, Val, Leu, Ile, Pro, Met and Trp, and genetically encoded hydrophilic amino acids include Thr, His, Glu, Gln, Asp, Arg, Ser, and Lys. Non-genetically encoded hydrophobic amino acids include t-butylalanine, while non-genetically encoded hydrophilic amino acids include citrulline and homocysteine.

Hydrophobic or hydrophilic amino acids can be further subdivided based on the characteristics of their side chains. For example, an aromatic amino acid is a hydrophobic amino acid with a side chain containing at least one aromatic or heteroaromatic ring, which may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR, etc., where R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe, Tyr, and Tryp, while non-genetically encoded aromatic amino acids include phenylglycine, 2-napthylalanine, beta-2-thienylalanine, 1,2,3,4-tetrahydroisoquinoline-3-carboxylic acid, 4-chlorophenylalanine, 2-fluorophenylalanine3-fluorophenylalanine, and 4-fluorophenylalanine.

An apolar amino acid is a hydrophobic amino acid with a side chain that is uncharged at physiological pH and which has bonds in which a pair of electrons shared in common by two atoms is generally held equally by each of the two atoms (i.e., the side chain is not polar). Genetically encoded apolar amino acids include Gly, Leu, Val, Ile, Ala, and Met, while non-genetically encoded apolar amino acids include cyclohexylalanine. Apolar amino acids can be further subdivided to include aliphatic amino acids, which is a hydrophobic amino acid having an aliphatic hydrocarbon side chain. Genetically encoded aliphatic amino acids include Ala, Leu, Val, and Ile, while non-genetically encoded aliphatic amino acids include norleucine.

A polar amino acid is a hydrophilic amino acid with a side chain that is uncharged at physiological pH, but which has one bond in which the pair of electrons shared in common by two atoms is held more closely by one of the atoms. Genetically encoded polar amino acids include Ser, Thr, Asn, and Gln, while non-genetically encoded polar amino acids include citrulline, N-acetyl lysine, and methionine sulfoxide.

An acidic amino acid is a hydrophilic amino acid with a side chain pKa value of less than 7. Acidic amino acids typically have negatively charged side chains at physiological pH due to loss of a hydrogen ion. Genetically encoded acidic amino acids include Asp and Glu. A basic amino acid is a hydrophilic amino acid with a side chain pKa value of greater than 7. Basic amino acids typically have positively charged side chains at physiological pH due to association with hydronium ion. Genetically encoded basic amino acids include Arg, Lys, and His, while non-genetically encoded basic amino acids include the non-cyclic amino acids omithine, 2,3,-diaminopropionic acid, 2,4-diaminobutyric acid, and homoarginine.

It will be appreciated by one skilled in the art that the above classifications are not absolute and that an amino acid may be classified in more than one category. In addition, amino acids can be classified based on known behaviour and or characteristic chemical, physical, or biological properties based on specified assays or as compared with previously identified amino acids. Amino acids can also include bifunctional moieties having amino acid-like side chains.

Conservative changes can also include the substitution of a chemically derivatised moiety for a non-derivatised residue, by for example, reaction of a functional side group of an amino acid. Thus, these substitutions can include compounds whose free amino groups have been derivatised to amine hydrochlorides, p-toluene sulfonyl groups, carbobenzoxy groups, t-butyloxycarbonyl groups, chloroacetyl groups or formyl groups. Similarly, free carboxyl groups can be derivatized to form salts, methyl and ethyl esters or other types of esters or hydrazides, and side chains can be derivatized to form O-acyl or O-alkyl derivatives for free hydroxyl groups or N-im-benzylhistidine for the imidazole nitrogen of histidine. Peptide analogues also include amino acids that have been chemically altered, for example, by methylation, by amidation of the C-terminal amino acid by an alkylamine such as ethylamine, ethanolamine, or ethylene diamine, or acylation or methylation of an amino acid side chain (such as acylation of the epsilon amino group of lysine). Peptide analogues can also include replacement of the amide linkage in the peptide with a substituted amide (for example, groups of the formula —C(O)—NR, where R is (C₁-C₆) alkyl, (C₁-C₆) alkenyl, (C₁-C₆) alkynyl, substituted (C₁-C₆) alkyl, substituted (C₁-C₆) alkenyl, or substituted (C₁-C₆) alkynyl) or isostere of an amide linkage (for example, —CH₂NH—, —CH₂S, —CH₂CH₂—, —CH═CH— (cis and trans), —C(O)CH₂—, —CH(OH)CH₂—, or —CH₂SO—).

The compound can be covalently linked, for example, by polymerisation or conjugation, to form homopolymers or heteropolymers. Spacers and linkers, typically composed of small neutral molecules, such as amino acids that are uncharged under physiological conditions, can be used. Linkages can be achieved in a number of ways. For example, cysteine residues can be added at the peptide termini, and multiple peptides can be covalently bonded by controlled oxidation. Alternatively, heterobifinctional agents, such as disulfide/amide forming agents or thioether/amide forming agents can be used. The compound can also be linked to a another compound that can modulate an apoptotic, aggregative, or proliferative response. The compound can also be constrained, for example, by having cyclic portions.

Peptides or peptide analogues can be synthesised by standard chemical techniques, for example, by automated synthesis using solution or solid phase synthesis methodology. Automated peptide synthesisers are commercially available and use techniques well known in the art. Peptides and peptide analogues can also be prepared using recombinant DNA technology using standard methods such as those described in, for example, Sambrook, et al. (Molecular Cloning: A Laboratory Manual. 2.sup.nd, ed., Cold Spring Harbor Laboratory, Cold Spring Harbor Laboratory Press, Cold Spring Harbor, N.Y., 1989) or Ausubel et al. (Current Protocols in Molecular Biology, John Wiley & Sons, 1994).

Compounds, such as peptides (or analogues thereof) can be identified by routine experimentation by, for example, modifying residues within htt proteins or polypeptides; introducing single or multiple amino acid substitutions, deletions, or insertions, and identifying those compounds that retain biological activity, e.g., those compounds that have the ability to modulate an apoptotic, aggregative, or proliferative response.

Compounds according to the invention include nucleic acid molecules, for example, cDNA, RNA, genomic DNA, or antisense molecules. Nucleic acid molecules can be used, for example, for gene therapy using standard techniques.

In general, candidate compounds for modulating cell survival, or for the prevention or treatment of cell degenerative or cell proliferative disorders are identified from large libraries of both natural product or synthetic (or semi-synthetic) extracts or chemical libraries according to methods known in the art. Those skilled in the field of drug discovery and development will understand that the precise source of test extracts or compounds is not critical to the method(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available. Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). In addition, natural and synthetically produced libraries of, for example, pancreatic beta cell precursor polypeptides containing leader sequences, are produced, if desired, according to methods known in the art, e.g., by standard extraction and fractionation methods. Furthermore, if desired, any library or compound is readily modified using standard chemical, physical, or biochemical methods.

When a crude extract is found to modulate cell survival, further fractionation of the positive lead extract is necessary to isolate chemical constituents responsible for the observed effect. Thus, the goal of the extraction, fractionation, and purification process is the careful characterization and identification of a chemical entity within the crude extract having cell proliferation, -preventative, or -palliative activities. The same assays described herein for the detection of activities in mixtures of compounds can be used to purify the active component and to test derivatives thereof. Methods of fractionation and purification of such heterogeneous extracts are known in the art. If desired, compounds shown to be useful agents for treatment are chemically modified according to methods known in the art. Compounds identified as being of therapeutic value may be subsequently analyzed using a mammalian HD model, or any other animal model for a degenerative or proliferative disorder.

Candidate test compounds can be first assayed for their ability to modulate the apoptotic, aggregative, proliferative or other response. The cells or cell preparations used in the assays can be obtained from cell lines or can be isolated from patients or animal models for degenerative or cell proliferative diseases or disorders, using standard techniques. The assays can be performed using standard assays as described herein, or known to those of ordinary skill in the art. Test compounds that modulate apoptotic, aggregative, proliferative responses can then be used for further analysis. Test compounds identified as being modulators of apoptotic, aggregative, or proliferative responses can be further tested in animal models of cell degeneration or proliferation, using standard techniques.

Antibodies

The compounds of the invention can be used to prepare antibodies to htt polypeptides or analogues thereof, using standard techniques of preparation as, for example, described in Harlow and Lane (Antibodies; A Laboratory Manual, Cold Spring Harbor Laboratory, Cold Spring Harbor, N.Y., 1988), or known to those skilled in the art. Antibodies can be tailored to minimise adverse host immune response by, for example, using chimeric antibodies contain an antigen binding domain from one species and the Fc portion from another species, or by using antibodies made from hybridomas of the appropriate species.

Pharmaceutical Compositions, Dosages, and Administration

Compounds of the invention can be provided alone or in combination with other compounds (for example, nucleic acid molecules, small molecules, peptides, or peptide analogues), in the presence of a liposome, an adjuvant, or any pharmaceutically acceptable carrier, in a form suitable for administration to humans. If desired, treatment with a compound according to the invention may be combined with more traditional and existing therapies for degenerative or proliferative diseases.

Conventional pharmaceutical practice may be employed to provide suitable formulations or compositions to administer the compounds to patients suffering from or presymptomatic for type 1 diabetes. Any appropriate route of administration may be employed, for example, parenteral, intravenous, subcutaneous, intramuscular, intracranial, intraorbital, ophthalmic, intraventricular, intracapsular, intraspinal, intracistemal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences” (19^{th} edition), ed. A. Gennaro, 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

For therapeutic or prophylactic compositions, the compounds are administered to an individual in an amount sufficient to stop or slow cell degeneration or cell proliferation, depending on the disorder. An “effective amount” of a compound according to the invention includes a therapeutically effective amount or a prophylactically effective amount. A “therapeutically effective amount” refers to an amount effective, at dosages and for periods of time necessary, to achieve the desired therapeutic result, such as reduction of cell death or apoptosis, or promotion of cell proliferation, for a cell degenerative disease, or the promotion of cell death or apoptosis, or reduction of cell proliferation, for a cell proliferative disease. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as reduction of cell death or apoptosis, or promotion of cell proliferation, for a cell degenerative disease, or the promotion of cell death or apoptosis, or reduction of cell proliferation, for a cell proliferative disease. Typically, a prophylactic dose is used in subjects prior to or at an earlier stage of disease, so that a prophylactically effective amount may be less than a therapeutically effective amount. A preferred range for therapeutically or prophylactically effective amounts of a compound may be 0.1 nM-0.1M, 0.1 nM-0.05M, 0.05 nM-15 μM or 0.01 nM-10 μM.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In the case of vaccine formulations, an immunogenically effect amount of a compound of the invention can be provided, alone or in combination with other compounds, with an adjuvant, for example, Freund's incomplete adjuvant or aluminum hydroxide. The compound may also be linked with a carrier molecule, such as bovine serum albumin or keyhole limpet hemocyanin to enhance immunogenicity.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

Assays

Various assays may be performed to determine biological activity of a test compound. For example, modulation of apoptosis, aggregation, or cell proliferation may be tested as described herein or as known by one of ordinary skill in the art.

Survival Assays

Screening for the pro-survival effect of a candidate compound, for example, a htt protein fragment can be performed using cell lines transfected with the gene encoding the candidate fragment. The transfected cells may be treated with a pro-apoptotic drug and one of several cellular markers of viability measured. These markers may include, without limitation: (1) cell death, measured by cell morphology; (2) mitochondrial viability, measured by enzyme activity; and (3) aggregate formation, measured by immunofluorescence staining. In vivo effects of htt in the transgenic mice can be assessed by: (1) observation of testicular cellular morphology by microscopy; and (2) DNA fragmentation using TUNEL staining. Additionally, cell lines can be created that stably express a biologically active fragment of htt that can be used as a reagent for screening the effectiveness of the fragment in protecting against multiple pro-cell death stimuli.

Cell Death or Apoptosis Assays.

Cells and samples may be obtained from a variety of sources including from experimental animals or human patients, from cell lines made using recombinant techniques, or from depositories such as ATCC. Alternatively, htt may be introduced into cells using standard transient transfection techniques.

Cells, for example, human neuronal precursor cell line NT2 cells, may be transfected with htt or control cDNAs using standard techniques. Cell death can be quantified in NT2 cells by co-transfection of the expression constructs with a plasmid containing a marker gene, for example, the LacZ gene at an appropriate ratio, and the cells may be stained for β-galactosidase activity at 24 hr post-transfection using standard procedures. The pro-survival effect of the candidate protein may be assessed by incubating transfected cells with a pro-apoptotic drug, for example, tamoxifen at various concentrations. An apoptotic morphology may be scored as blue-staining cells that are rounded up, blebbed and condensed, which may be clearly distinguished from viable cells that are flat and have neurite-like extensions.

Mitochondrial Viability

Cells, for example, HEK 293T cells may be transfected with htt or a control DNA, for example, LacZ DNA using standard techniques. Transfected cells may be treated at 48 hours post-transfection with a various concentrations of tamoxifen or other apoptosis inducer. Cell viability may measured by MTT assay at 24 hr post-transfection by incubating the cells for 2 hr in a 1:10 dilution of WST- 1 reagent (Boehringer Mannheim) and release of formazan from mitochondria may be quantified at 450 nm using an ELISA plate reader. Mock transfected, vector only, and LacZ transfected cells serve as controls for transfection-related toxicity. One way ANOVA and Newman-Keuls test may be used for statistical analysis. Statistical analyses of the cell death data in NT2 and HEK cells may be performed using one-way ANOVA and Newman-Keuls post-comparison tests.

Aggregate Formation.

Cells, for example, human embryonic kidney cells (HEK 293T) may be transfected using standard techniques. At 48 h post-transfection, the cells may be treated with tamoxifen to induce aggregate formation, then processed for immunofluorescence. The cells can be fixed, permeabilised, then incubated with anti-htt antibody. Secondary antibodies conjugated to a marker, such as FITC with the use of DAPI (4′,6′-diamindino-2-phenylindole) as a nuclear counter-stain. Appropriate control experiments may be performed to determine the specificity of the antibodies, including secondary antibody only and mock transfected cells. The cells may be viewed with microscope, digitally captured with a CCD camera and the images may be colourised and overlapped. The proportion of cells with aggregates is presented as a percent of the total number of cells expressing htt.

Cell Proliferation Assays

Various cell proliferation assays, such as those described herein or known to one of ordinary skill in the art may be use. Such assays include MTT assays; contact inhibition assays, conducted on soft agar or in an animal model, for example, to determine tumour growth; as well as multiple commercially-available cell proliferation assays.

Animal Models

Various animal models of cell degenerative or cell proliferative diseases exist. In addition to the YAC transgenic htt model described herein, the familial ALS-SOD1 transgenic mouse model may be used as an amyotropic lateral sclerosis model; the rds mouse may be used as a model for retinal degeneration; the EAE mouse may be used as an animal model of human multiple sclerosis; etc. These animal models may be used as source cells or tissue for the assays of the invention. Test compounds may also be assayed in these models.

The following examples are intended to illustrate various embodiments and aspects of the invention, and do not limit the invention in any way.

### GENERAL METHODS USED

Generation of Experimental Mice

Heterozygous knockout (KO) mice that had only one copy of the wild-type htt allele were bred with YAC transgenic mice to generate a series of F1 generation mice that express the YAC transgene on a background that is heterozygous for endogenous htt (“YAC rescued mice”). More specifically, YAC transgenic mice (FVB/NJ strain) from lines 29 (YAC18), 668 (YAC46), and 2511 (YAC72) were bred with mice heterozygous for targeted disruption of the endogenous mouse Hdh gene (C57BL6 strain) to produce F1 generation hybrid mice. The YAC transgenes used in these studies contained 18, 46 or 72 CAG repeats. F1 hybrid mice positive for the YAC transgene and heterozygous for the Hdh gene were then bred to generate the experimental F2 mice with the following genotypes: YAC transgene positive or negative on a background of normal endogenous htt (+/+), half normal endogenous htt (+/−), and lacking endogenous htt (−/−) (Hodgson et al. (1996) Hum. Mol. Genet. 5, 1875-1885). All the F2 offspring of these matings were genotyped and used to generate experimental data. Selected F2 mice were also bred to examine mating behavior, breeding success rates and to obtain post-coital sperm counts. Genotyping was performed by standard PCR based techniques on genomic DNA from tail clippings prepared by phenol-chloroform extraction (Hodgson et al. (1996) Hum. Mol. Genet. 5, 1875-1885). Protein expression was determined by Western blot in which 200 ug of total protein from homogenized testes was loaded onto a low-bis acrylamide gel, run at 100V for 2 hours, and 200V for 3 hours before being transferred to PVDF membranes. Blots were probed with anti-htt antibody (HD3 @ 1/1000, Gutekunst et al. (1999) J. Neurosci. 19, 2522-2534) and detected using ECL (Amersham).

Fertility, Mating Behavior, and Sperm Analysis

To assess breeding success, adult male mice of each genotype were placed in breeding cages with single FVB/NJ female mice for at least four months (minimum of three male mice tested per genotype). The total duration of time spent in breeding cages and the total number of live-born litters was recorded for each mouse. Failure to produce any litters after a cumulative duration of breeding of at least 4 months was considered to represent male infertility. New wild-type females with previous successful breeding experience were placed in breeding cages of infertile males to control for any contribution of the female partner. YAC72−/− (mice expressing human htt with 72 polyglutamines and no endogenous murine htt), YAC72+/− (mice expressing human htt with 72 polyglutamines on a background of half the normal levels of endogenous murine htt), and YAC72+/+ (mice expressing human htt with 72 polyglutamines on a background of normal levels of endogenous murine htt) male mice were placed in breeding cages with pseudo-pregnant FVB/NJ females to assess male sexual behavior and post-coital sperm counts. These wild-type female mice were injected with 0.1 ml of pregnant mare serum (Sigma) 48 hours prior to stimulation with 10 IU of hcg (Sigma) and placement in breeding cages with the transgenic males. Mounting behavior of male mice was scored for 2-3 hours following placement in breeding cage and plug formation was determined by manual inspection of the female mice the following morning.

Post-coital sperm counts were determined from the extracted uterus of all female mice who had evidence of plug formation following breeding with male transgenic mice. Following breeding the plugged female mice were anaesthetized, the uterus and oviducts removed in toto and gently opened in a sterile 12 well tissue culture plate. 0.5 ml of sterile saline was used to flush the uterus, the resultant solution was collected, and examined microscopically for presence of sperm. To obtain quantitative sperm counts, the testes and epidydimi of YAC72+/+, YAC72+/−, and YAC72−/− mice (n=6 mice per genotype) were removed and immediately weighed. The tissues were then sectioned and placed in tubes containing 0.5 ml of sterile saline. Total numbers of morphologically normal sperm were manually counted using a brightline hemacytometer (Hausser) for three samples from each tissue. The hemacytometer counts for each tissue were averaged, and the total counts per ml were calculated. Results are expressed as average sperm count per ml +/+ SEM and significance was determined by the Students t-test.

Histological and Ultrastructural Analysis

Testes were removed from anesthetized animals and immediately placed in fixative (1.5% paraformaldehyde, 1.5% glutaraldehyde, 0.1 M Na cacodylate, pH 7.3). The capsules were nicked with a scalpel and then the organs left to fix for approximately 1 hour. The testes were cut into small pieces and fixed for an additional hour. The pieces were washed with buffer, post-fixed in buffered 1% OSO₄ on ice for 1 hour, washed with dH₂O, and stained en bloc in 1% aqueous uranyl acetate. The samples were washed with dH₂O, dehydrated through a graded concentration series of ethyl alcohols, and then embedded in JEMBED™812 (J. B. EM Services Inc., Point-Claire, Quebec).

For histological analysis, thick sections (1 μm) were stained with toluidine blue and evaluated on a Zeiss Axiophot microscope. For ultrastructural analysis, thin sections were cut on an ultramicrotome, stained with lead citrate and uranyl acetate, and then viewed and photographed on a Philips 300 electron microscope operated at 60 kV.

TUNEL™ labeling (Boehringer Mannheim) was performed using standard techniques on frozen sections of testes that were lightly immersion fixed in 3% paraformaldehyde. Following removal of the testes, mice were injected with heparin and transcardially perfused with 3% paraformaldehyde and 0.15% glutaraldehyde in phosphate buffer (pH 7.4). Brains were then removed and post-fixed in 3% paraformaldehyde overnight.

Immunocytochemistry

Testes were excised from anesthetized animals, the capsules cut open with a scalpel, and the organs immersion fixed (3% paraformaldehyde, 150 mM NaCl, 5 mM KCl, 3.2 mM Na₂HPO₄, 0.8 mM KH ₂PO₄, pH 7.3) for one to two hours. Following fixation, the testes were washed three times (10 minutes each wash) with PBS (150 mM NaCl, 5 mM KCl, 3.2 mM Na.sub.2HPO.sub.4, 0.8 mM KH₂PO₄, pH 7.3) and then frozen in OCT compound and sectioned on a cryostat. Sections (10 μm thick) were collected on polylysine-coated slides and then the slides were immediately plunged into cold acetone (−20° C.) for 5 minutes. Following this, the slides were air-dried.

Sections were re-hydrated for 30 minutes in TPBS (PBS, 0.05% Tween-20, 0.1% BSA) containing 5% normal goat serum (NGS), and then incubated for 1 hour at 37° C. with primary antiserum diluted 1:100 (HD3) with TPBS containing 1% NGS. Sections were washed (three times 10 minutes each wash) with TPBS, and then incubated for 1 hour at 37° C. with secondary antibody (goat anti-rabbit conjugated to Texas red) diluted 1:100 in TPBS. Sections again were washed with TPBS, mounted in Vetashield™ (Vector Laboratories, Burlingame, Calif.), and viewed on a Zeiss Axiophot microscope fitted with the appropriate fluorescence filter sets. Controls consisted of replacing the primary antibody with the same concentration of normal rat IgG (control for primary antibody), replacing the primary antibody with buffer alone (control for secondary antibody), and replacing the primary and secondary antibodies with buffer alone (control for autofluorescence).

### EXAMPLE I

Offspring from Crosses of YAC Transgenic Mice

Mice of each genotype of interest were set-up with FVB/NJ wild-type mates and allowed to remain in breeding cages for a minimum of 4 months with the number of pregnancies, litters, and pups recorded. Several breeding pairs were set-up per genotype and the results of a minimum of 20 months of combined breeding time tabulated per genotype.

Table 1 demonstrates the outcome of crosses between YAC transgenic mice that are heterozygous for the Hdh null allele (htt knockout mice). The total number of live-born offspring is given for each transgenic line. Note that the offspring have the expected 1:2:1 ratio indicating that there was no significant foetal loss and that the human transgene is capable of rescuing the Hdh nullizygous state, indicating that the transgene has normal htt developmental expression and function.

TABLE 1
Offspring of HD Knock-out Rescue Breedings

| | +/+ | +/− | −/− |
|---|---|---|---|
| YAC18 | 19 | 33 | 15 |
| YAC46 | 17 | 60 | 31 |
| YAC72 | 9 | 8 | 4 |
| Total | 45 | 101 | 49 |

In Vivo Effect of Reduced Expression of Normal Htt

Mice were bred to be transgenic for human htt and to express various levels of wild-type mouse htt, as described herein, to determine the in vivo effect of reduced expression of normal htt. The breeding strategy allowed the generation of mice expressing htt with different CAG repeat sizes, with or without co-expression of normal htt.

The cellular effect of the htt expression was determined by analyzing the testicular morphology of YAC rescued htt knockout mice. Whole testes were removed from 8 month old adult male mice of each genotype. For semi-thin toluidine blue staining, testes were cut in 40 μm coronal sections using a vibratome, collected in PBS, osmicated (1% OSO₄ in 0.1M cacodylate buffer), rinsed, and stained overnight in 2% aqueous uranyl acetate. All the sections used were dehydrated in ascending concentrations of ethanol and propylene oxide (1:1) and flat embedded in Eponate 12 Semi-thin sections (1.5 μm) were cut using a Leica Ultracut S ultramicrotome, counterstained with Toludine Blue or Cresyl Violet, differentiated in 95% alcohol and coverslipped. Sections were visualized using a Nikon Microphot FXA equipped with a 60× oil immersion lens.

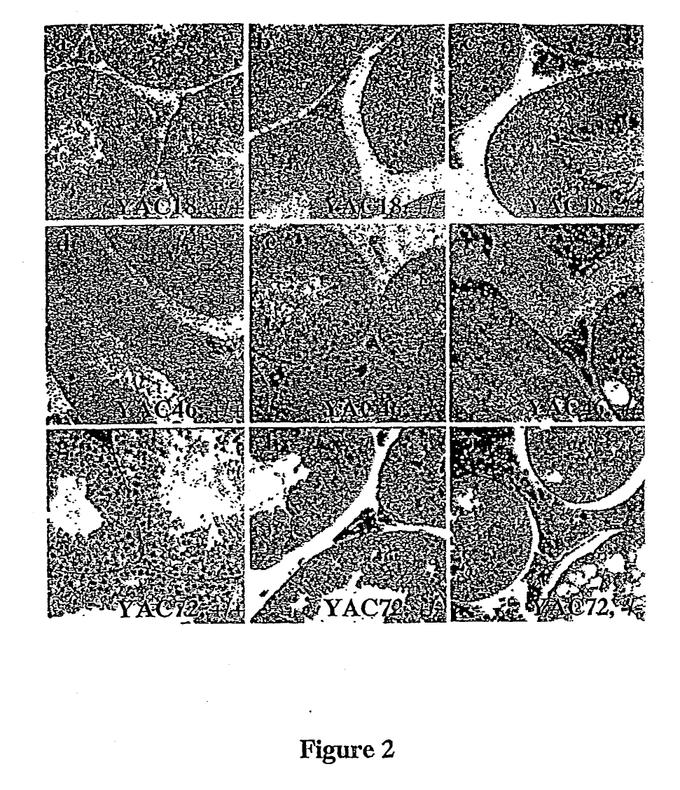

FIGS. 2A-I show massive degeneration of spermatocytes. This cell death phenotype is CAG repeat size-dependent and is modulated by the level of endogenous htt. The cell death is most pronounced in YAC72 rescued knockout mice, intermediate in YAC46 knockout mice, and not present in YAC 18 rescued mice.

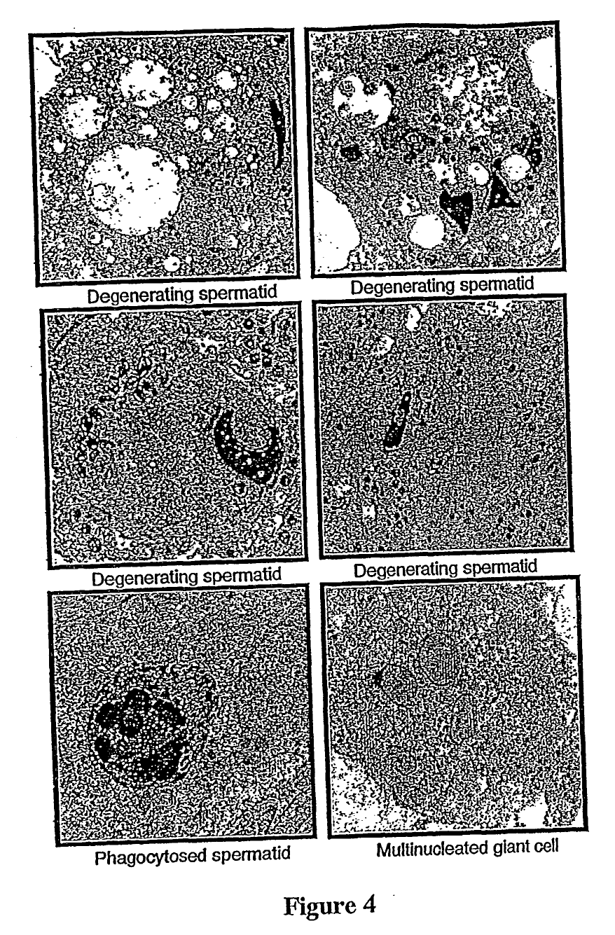

Electron microscopic analysis was also used to further characterize the degeneration phenotype in the YAC72 mice lacking endogenous htt (FIG. 4). Ultrastructural analysis of testes from YAC72 mice nullizygous for endogenous htt revealed massive cell death of spermatids, phagocytosis of degenerating cells, and formation of multinucleated giant cells. These data demonstrate the role of endogenous htt in protecting against mutant htt-induced degeneration.

Increased Apoptosis with Mutant Htt

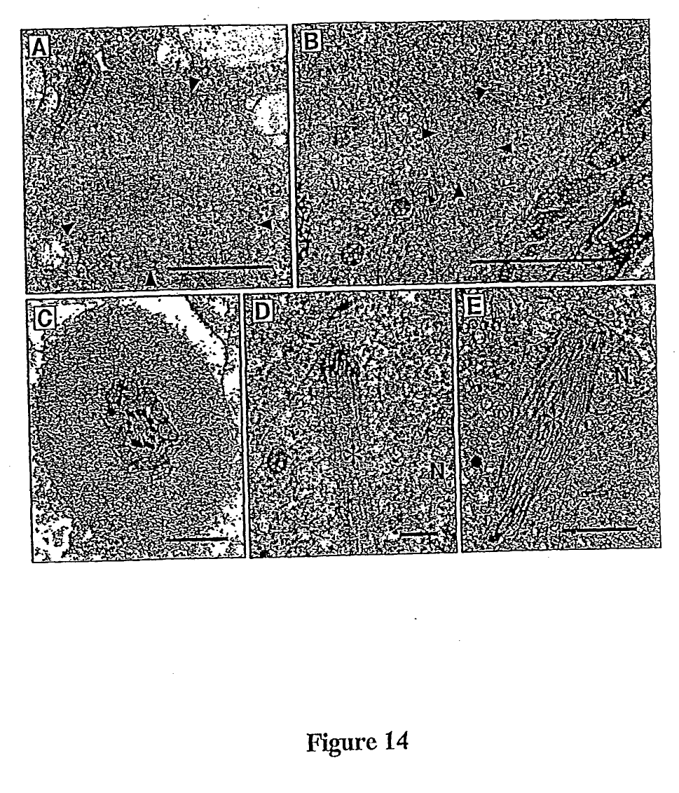

TUNEL™ staining was used to quantitate apoptosis in the YAC rescued transgenic mice. Whole testes were removed from adult mice of each genotype. For TUNEL™ analyses, the testes were immersion-fixed overnight in paraformaldehyde, cryopreserved in sucrose solution, frozen, and cryostat sectioned at 10 μm. For immunocytochemistry, slide mounted sections were incubated in blocking solution for one hour and then in diluted primary antibody solution overnight. After serial washes in phosphate buffered saline (PBS), the sections were incubated in diluted secondary antibody for two hours, washed, and mounted under coverslips. TUNEL™ labeling was performed using standard techniques on frozen sections of testes that were lightly immersion-fixed in 3% paraformaldehyde, using the in situ cell death detection kit (Boehringer Mannheim), according to the manufacturer's instructions. These experiments demonstrated increased apoptosis in the testes of YAC72 rescued htt knockout mice, indicating that absence of endogenous htt leads to cell death (FIG. 14). 13?

### Example 5

### Pro-Survival and Anti-Apoptotic Effects of Htt Tamoxifen-Induced Cell Death

The survival effect of htt was measured in vitro using the MTT assay, in which a reduction in mitochondrial viability is indicative of cell death. Human embryonic kidney (HEK) 293T cells were seeded at a density of 5×10⁴ cells into 96-well plates and transfected with 0.1 μg htt or LacZ DNA using a standard calcium phosphate protocol (Hackam et al. (1998) J Cell Biol. 141: 1097-105). Transfected cells in 96-well plates were treated at 48 hours post-transfection with a various concentrations of tamoxifen, a cell permeable compound that leads to caspase activation and cell death, for 4 hours. Cell viability was measured, at 24 hours post-transfection, by incubating the cells for 2 hours in a 1:10 dilution of WST-1 reagent (Boehringer Mannheim) and release of formazan from mitochondria was quantified by the MTT assay at 450 nm using an ELISA plate reader (Dynatech Laboratories). Mock transfected, vector only and LacZ transfected cells served as controls for transfection-related toxicity. Statistical analyses of the cell death data were performed using one-way ANOVA and Newman-Keuls post-comparison tests.

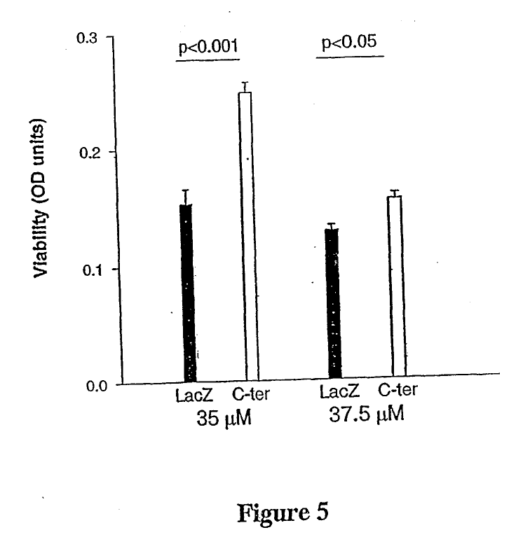

The results indicated that expression of the C-terminus (C-ter) of htt protects HEK 293T cells from tamoxifen-induced cell death, when compared with expression of the β-galactosidase (LacZ) control (FIG. 5).

Mutant Htt-Induced Cell Death

An additional cell line was tested with a different cell death induction paradigm, to demonstrate that the C-terminus protection is not specific to tamoxifen-induced cell death. Mutant htt protein (HD138) was co-transfected with control protein, pyruvate kinase (PK), or with htt C-terminus (C-ter) into NT2 cells (a human neuronal precursor cell line) at 40% density using lipofectamine (GibcoBRL), and cell death in response to tamoxifen stimulus was measured. Cell death was quantified in NT2 cells by co-transfection of the expression constructs with a plasmid containing the LacZ gene at a 4:1 ratio, and the cells were stained for β-galactosidase activity at 24 hours post-transfection using standard procedures. The survival effect of the htt protein was assessed by incubating transfected cells with the pro-apoptotic drug tamoxifen for 4 hrs at various concentrations. An apoptotic morphology was scored as blue-staining cells that were rounded up, blebbed and condensed, which were clearly distinguished from viable cells that were flat and had neurite-like extensions. The cell death data was analyzed for statistical significance using one-way ANOVA and Newman-Keuls post-comparison tests.

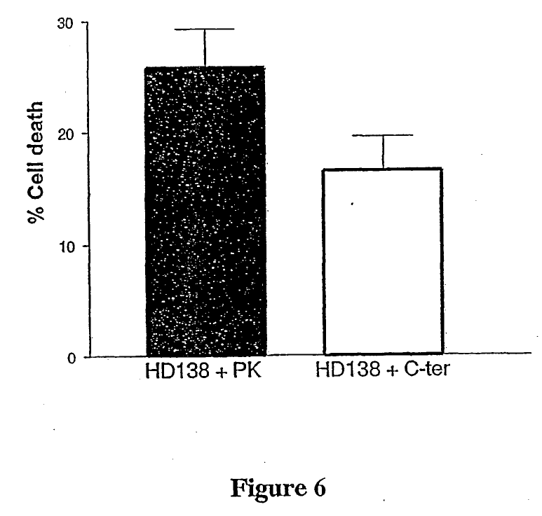

Expression of the C-terminus reduced HD138-dependent cell death (p=0.002) (FIG. 6). The results thus indicate that the C-terminus of htt confers protection against mutant htt-induced toxicity in NT2 cells. Furthermore, this data supports the in vivo evidence that wild-type htt is essential for protecting against mutant htt in the YAC72 mice.

HIP-1-Induced Cell Death

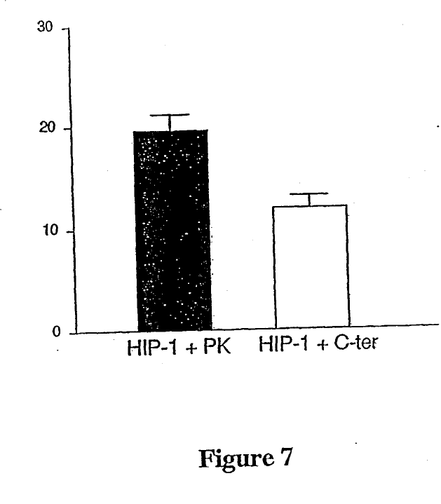

The htt interacting protein HIP-1 is a pro-apoptotic protein that rapidly induces cell death in a caspase-dependent manner, and toxicity is exacerbated in the presence of mutant htt, suggesting that HIP-1 may be involved in HD pathogenesis. HIP-1-induced toxicity was used to assess whether the C-terminus of htt confers protection against a toxic protein-mediated cell death, in addition to its effect on tamoxifen-induced death. NT2 cells were co-transfected with mutant HIP-1 and the C-terminus, or HIP-1 and the control pyruvate kinase cDNA, using lipofectamine (GibcoBRL). Cell death was measured by morphological changes using co-transfection of a plasmid containing the LacZ gene, as described herein. Expression of htt C-terminus in NT2 cells reduced HIP-1 mediated cell death, compared with expression of control protein (PK) (p<0.01) (FIG. 7). The results indicated that expression of htt C-terminus in NT2 cells reduced cell death induced by HIP-1. Thus, the protective activity of htt C-terminus may have a functional role in HIP-1-mediated cell death in vivo.

Abnormal Protein Aggregation

Mutant polyglutamine containing proteins are known to aggregate into large amorphous protein clumps. Mutant htt protein forms aggregates in HD brains, transgenic mice and in cell culture. Truncated and full-length htt containing 128 CAG repeats readily forms aggregates in HEK 293T cells when the cells are exposed to apoptotic stress by tamoxifen (Hackam et al. (1998) J Cell Biol. 141:1097-105). In the YAC72 transgenic mice, htt aggregates were observed in the striata late in the pathological process (Hodgson et al. Neuron 1999, 23:181-92). Therefore, htt aggregates are markers for pathological changes in vivo and in vitro, and can be used as an additional marker of cell viability.

Ultrastructural analysis using electron microscopy was used to determine the effect of endogenous htt on the formation of aggregates. Testes were cut in 40 μm coronal sections using a vibratome, collected in PBS, osmicated (1% OSO₄ in 0.1M cacodylate buffer), rinsed, and stained overnight in 2% aqueous uranyl acetate. All the sections used were dehydrated in ascending concentrations of ethanol and propylene oxide (1:1) and flat embedded in Ultrathin sections (90 nm) were cut using a Leica Ultracut S ultramicrotome, counterstained with 5% aqueous uranyl acetate for 5 minutes followed by lead citrate for 5 minutes. Thin sections were examined using a HITACHI H-7500 electron microscope.

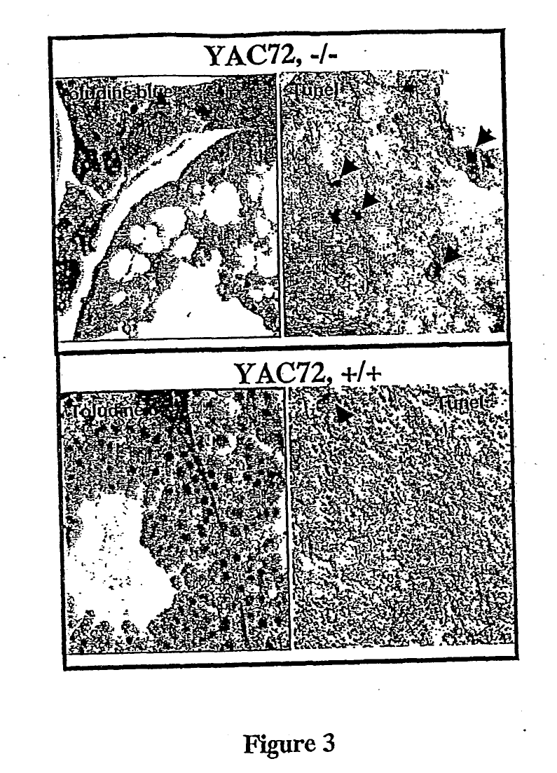

Ultrastructural analysis of the testes of YAC72 mice lacking endogenous htt revealed the presence of abnormal aggregates of intracellular protein (arrows) within spermatids, sertoli cells and sperm tails (FIG. 3). Ectopic microtubule bundles and manchettes were also identified (arrows). The protein aggregates resemble the ultrastructural appearance of htt aggregates found in human HD brain tissue.

Reduction of Mutant Htt Aggregation by C-Terminus of Htt

The C-terminus of htt was co-transfected with mutant htt and aggregate formation was induced with tamoxifen or HIP-1 expression as described herein. HEK 293T cells were transfected with truncated mutant htt with 128 CAG repeats and processed for immunofluorescence by growing cells on glass coverslips and transfecting at 30% confluency using a standard calcium phosphate protocol. At 48 hours post-transfection, the cells were treated with 35 μM tamoxifen (Sigma) for 1 hour to induce aggregate formation, then processed for immunofluorescence. The cells were fixed in 4% paraformaldehyde/PBS, permeabilised in 0.5% Triton X-100/PBS for 5 min, then incubated with anti-htt antibody MAB2166 (Chemicon) (1:2500 dilution) diluted in 0.4% BSA/PBS. Secondary antibodies conjugated to FITC (Jackson Laboratories) were used at 1:600-1:800 dilutions, and DAPI (4′,6′-diamindino-2-phenylindole, Sigma) was used as a nuclear counter-stain. Appropriate control experiments were performed to determine the specificity of the antibodies, including secondary antibody only and mock transfected cells. The cells were viewed with a Zeiss (Axioscope) microscope, digitally captured with a CCD camera (Princeton Instrument Inc.) and the images were colourised and overlapped using the Eclipse (Empix Imaging Inc.) software program. The proportion of cells with aggregates is presented as a percent of the total number of cells expressing htt.

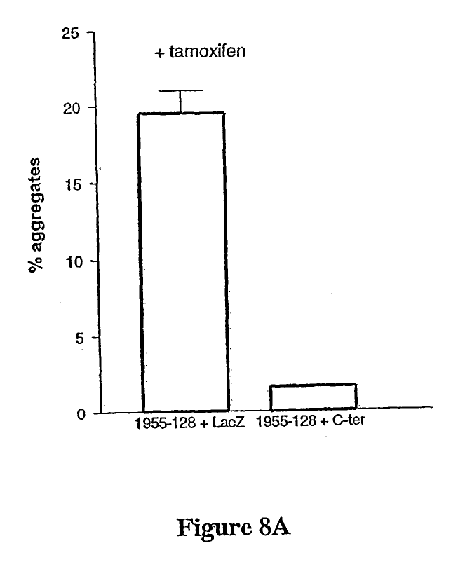

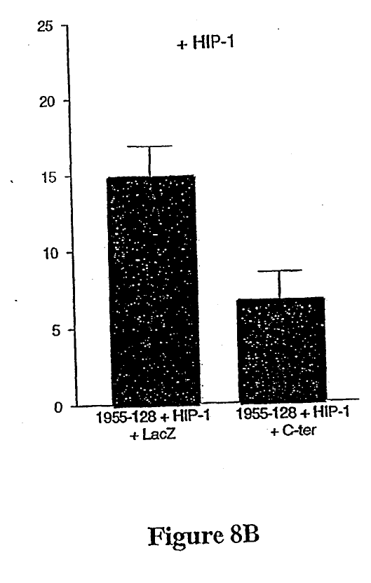

Expression of the htt C-terminus (C-ter) reduces the number of aggregates formed by a truncated version of mutant htt protein (1955-128), compared with coexpression of htt and the LacZ control (FIGS. 8A-B). Aggregates were induced by tamoxifen stimulus FIG. 8A) or by HIP-1 expression (FIG. 8B). The data indicate that the C-terminus of htt protein is able to reduce mutant htt protein aggregate formation in transfected cells.

Over-Expression of Full-Length Wild-Type Human Htt in Mice Confers Protection Against Excitotoxic Neurodegeneration

Yeast artificial chromosome (YAC) transgenic mice were generated that over-express wild-type human htt (line 212) at 2-3 times the levels of endogenous htt in wild-type mice (FVB/NJ) (Hodgson et al.(1996) Hum. Mol. Genet. 5, 1875-1885). Fifteen of these transgenic and 23 control FVB/NJ mice 4-8 months of age each received a single intraperitoneal injection of 25 mg/kg kainic acid (KA) or an equal volume of vehicle (PBS). Intraperitoneal injections of kainic acid injections cause prolonged seizures in mice, and each mouse was observed continuously for two hours following injection. The occurrence, severity, and duration of seizures was recorded.

One week following KA injection, brains were removed from one group of the mice and immediately frozen in isopentene on dry ice. Serial 30 μm coronal cryostat sections were cut through the entire hippocampus. After every fifth section, two sections were removed for quantitative analysis and fixed in 3% paraformaldehyde for 30 minutes in preparation for histochemical staining. Degenerating neurons were identified in hippocampal sections by Fluoro-Jade™ histochemistry (Histo-Chem Inc.), silver staining (FD Neurotechnologies) and by TUNEL™ (Boehringer Mannheim). Fluoro-Jade™ is a fluorescent stain that labels degenerating neurons in fixed brain sections. The total number of degenerated hippocampal neurons labeled with Fluoro-Jade™ was recorded from the CA1, CA3, and total hippocampus regions of each section selected for quantitative analysis. Slide-mounted sections were viewed with a Zeiss (Axiovert) fluorescent microscope, digital photomicrographs were captured with a cooled CCD camera (Princeton), and degenerating hippocampal neurons manually counted in a blinded fashion. To assess the role of caspase activation in this process, the hippocampus and cerebellum were removed from a second group of animals 8 hours following KA-induced seizures. Caspase activity was measured in homogenized hippocampal and cerebellar samples using the fluorogenic substrate acetylated DEVD aminofluorocoumarin. DEVD-ase activity was standardized to protein content as determined by standard Lowry analysis.

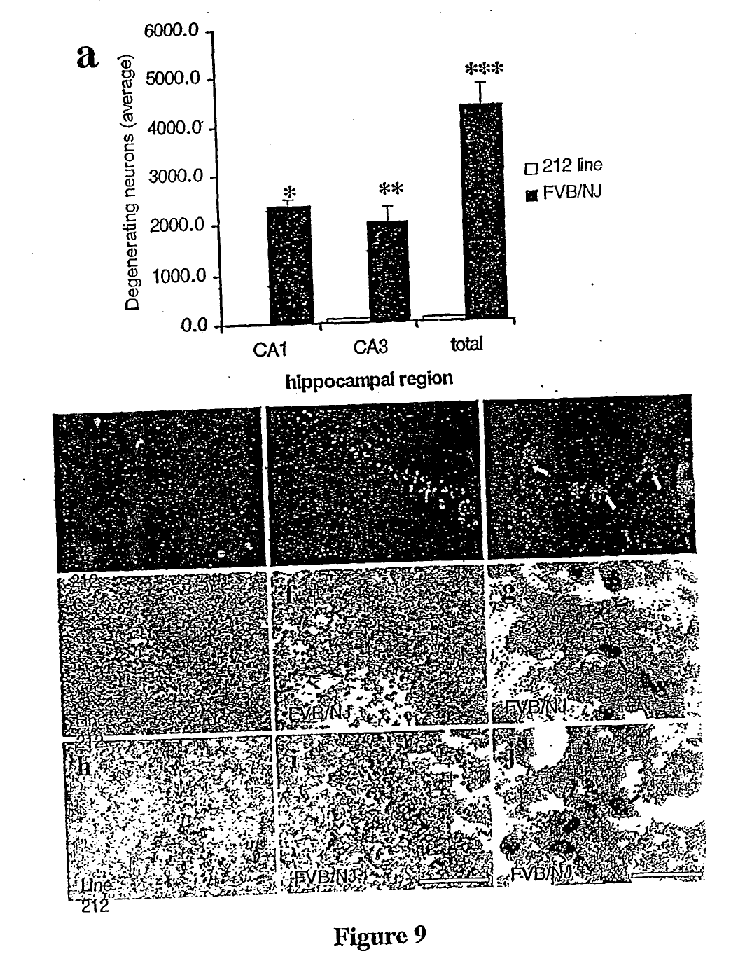

Following KA-induced seizures YAC transgenic mice expressing 2-3 times the endogenous levels of wild-type htt averaged approximately 50-fold less (81 vs. 4362, * * * p<0.0001) degenerating hippocampal neurons than control animals (FIG. 9A). Fluoro-jade brightly labeled the soma and large processes of degenerating hippocampal neurons within CA1 and CA3 of KA-treated transgenic and wild-type mice (FIGS. 9B-D). Degenerating neurons were predominantly restricted to the CA1 and CA3 regions of hippocampus. KA-induced neurodegeneration was significantly reduced in both the CA1 (3 vs. 2360, * P<0.000001) and CA3 regions (79 vs. 2003, ** P<0.0003) of transgenic relative to control mice. Intraperitoneal injection of 25 mg/kg of kainic acid was sufficient to cause prolonged seizures in all the mice in this study irrespective of genotype. No seizure activity or neurodegeneration was observed in any mouse following injection of PBS.

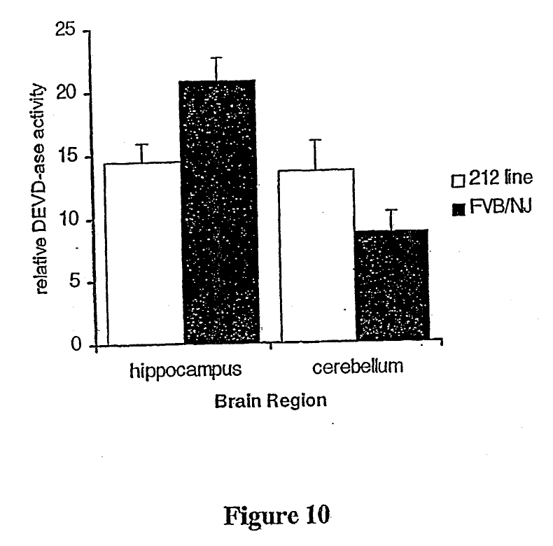

Argyrophyllic labelling of neuronal soma was dramatically reduced in adjacent silver-stained hippocampal sections from transgenic (FIG. 9E) compared to wild-type mice (FIGS. 9F and 9G) following KA-induced seizures, confirming the results of Fluoro-Jade™ staining using this well-established marker for neurodegeneration. TUNEL™ staining (FIG. 9H) labeled few apoptotic hippocampal neurons following KA-induced seizures in brains from transgenic mice, but many TUNEL™ labeled neurons were identified in wild-type brains (FIGS. 9I and 9J). Significantly less hippocampal caspase activation was evident in transgenic mice compared to wild-type mice following KA-induced seizures (FIG. 10, 14.5 +/−1.49 vs. 20.8 +/−1.77, * P=0.02) in parallel to the observed effects of wild-type htt on neurodegeneration. No significant difference was found in cerebellar caspase activation for transgenic vs. wild-type (FIG. 10, 13.66 +/−2.32 and 8.68 +/−1.67). Degeneration does not occur in the cerebellum following KA-induced seizures.

Thus, transgenic mice expressing 2-3 times endogenous levels of wild-type htt were resistant to apoptotic neurodegeneration following kainic acid-induced seizures, having approximately 50-fold less kainic acid-induced hippocampal neurodegeneration than littermate controls. Caspase activity levels within the hippocampus were increased following KA-induced seizures, and there was less DEVD-ase activation in the presence of increased levels of wild-type htt. These data demonstrate a significant anti-apoptotic role for wild-type htt in neurons of the central nervous system. This anti-apoptotic effect of wild-type htt acts upstream of caspase activation. The DEVD-ase fluorogenic assay captures the enzymatic activity of caspase-2, −3, and −7, and does not identify the specific caspases activated in KA-induced seizures. Modulation of caspase-dependent pathways by wild-type htt may alter the sensitivity of neurons to excitotoxic stress. These results further demonstrate that there is a relationship between normal htt function and neuronal susceptibility to excitotoxicity.

Wild-Type Htt Reduces the Cellular Toxicity of Mutant Htt In Vivo

Mutant human htt causes increased apoptotic cell death in the testes of transgenic mice expressing no endogenous htt. This pro-apoptotic effect of mutant htt can be completely inhibited by increased levels of murine wild-type htt, indicating that wild-type htt can reduce the toxicity of mutant htt.

Rescue of the Hdh−/−Lethal Phenotype by YAC Transgenes Expressing Mutant Human Htt

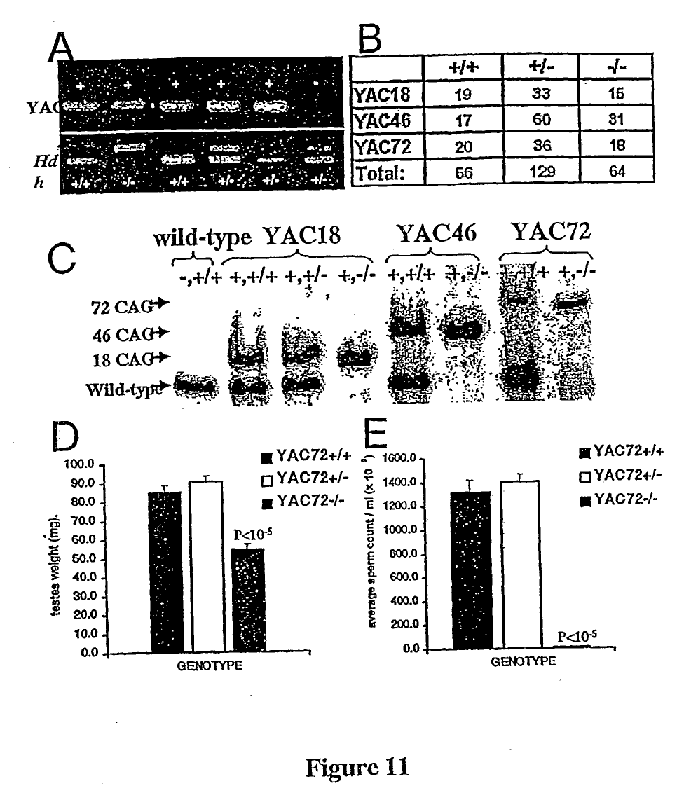

YAC transgenic mice expressing normal (YAC18) or mutant (YAC46 or YAC72) human htt in the absence of endogenous mouse htt (Hdh−/−) were generated. FIG. 11A demonstrates the genotype of several offspring from a cross between two mice heterozygous for targeted disruption of the Hdh gene, one of which also carried the YAC72 transgene (YAC72+/−). Mice with targeted disruption of both alleles of the Hdh gene can be rescued from the embryonic lethal phenotype by the YAC transgene expressing mutant htt with 72 CAG repeats (YAC72−/− mice). In this litter, mice were generated with the YAC72 transgene and either 100% of the normal level of endogenous htt (YAC72+/+), 50% of the normal level of endogenous htt (YAC72+/−), or complete absence of endogenous htt (YAC72−/−). A mouse lacking the YAC72 transgene but heterozygous for endogenous htt is also shown (FIG. 11A, −,+/−). No Hdh−/− mice were generated in the absence of the YAC transgene, consistent with the previous finding that Hdh nullizygous mice are not viable (Nasir et al. (1995) Cell 81, 811-823; Duyao et al. (1995) Science 269, 407410; Zeitlin et al. (1995) Nat. Genet. 11, 155-162). The F2 offspring of our experimental breedings had the expected 1:2:1 ratio of genotypes for all of the YAC transgenes examined (FIG. 11B), demonstrating that both the normal (YAC18) and mutant (YAC46 and YAC72) human HD transgenes compensated for the lack of endogenous murine htt in Hdh-/- mice equally. Thus, mice with targeted disruption of the Hdh gene were rescued from the embryonic lethal phenotype equally by all three of the YAC transgenes.

Htt Expression Levels in YAC Transgene Rescued Hdh−/− Mice

Levels of the transgenic and wild-type htt protein for YAC 18 (line 29), YAC46 (line 668), and YAC72 (line 2511) Hdh−/− mice were measured by Western blot using an antibody recognizing both human and mouse htt (Hodgson et al. (1996) Hum. Mol. Genet. 5, 1875-1885). The human transgenic protein is expressed at similar levels in the YAC18, YAC46, and the YAC72 mice used in these experiments (FIG. 11C). This result was replicated in three different Western blots, and densiometric quantification of the transgenic protein level from these blots revealed a 1:1 ratio of transgenic versus endogenous htt levels in these mice. Transgenic protein levels from YAC18 mice averaged 102% of wild-type htt, YAC46 averaged 99% of wild-type htt, and YAC72 averaged 100% of wild-type htt. These 3 lines of YAC transgenic mice were selected for these experiments because they expressed identical levels of transgenic protein, differing only in the length of the CAG repeat.

Mice Expressing Mutant Htt are Infertile

Expression of mutant (YAC46 and YAC72), but not wild-type (YAC18), transgenic htt in the absence of endogenous htt leads to a novel phenotype that was initially identified by the observation that male Hdh −/− mice were unable to breed. We attempted to breed YAC46−/− and YAC72−/− males with wild-type females for extended periods with no success (no offspring were generated). Female littermates with the same genotype (YAC46−/− and YAC72−/−) had normal fertility when bred with wild-type mice. Male YAC72−/− mice had normal secondary sexual characteristics and displayed identical sexual behavior (mounting) and libido as YAC72+/− and YAC72+/+ mice when placed in cages with female wild-type mice. Similar plug formation rates were obtained for males of all three genotypes. Plugs were recovered in 8 of 12 breeding trials for YAC72-/- mice, 7 of 12 breeding trials for YAC72+/− mice and 8 of 16 breeding trials of YAC72+/+ mice. In sharp contrast to both YAC72+/+ and YAC72+/− mice, no post-coital sperm was ever recovered from plugged females following mating with YAC72−/− mice. These results suggested that a defect in spermatogenesis and not breeding behavior was responsible for the observed lack of fertility of male YAC72−/− mice.

Decreased Fertility in Mice Expressing Mutant Htt is a Result of Decreased Sperm Production

To assess spermatogenesis directly in YAC72−/− mice, we performed sperm counts and examined the testes of these mice. YAC72−/− mice had significantly decreased epidydimal sperm counts compared to YAC72+/+ (11×10³/ml +/−2.9 vs. 13×10⁵/ml +/−100; p<0.0000l) or YAC72+/− (11×10³/ml+/−2.9 vs 14×10⁵/ml +/−65; p<0.0000l) littermates at 4 months of age (FIG. 11D).

Testicular Atrophy and Spermatid Degeneration in Mice Expressing Mutant Htt

YAC72−/− mice had significant testicular atrophy compared to YAC72+/+ (average testes weight, 54.0 mg +/−2.9 vs. 84.7 mg +/−3.6.9; p<0.00001) and YAC72+/− (average testes weight, 54.0 mg +/−2.9 vs. 90.1 mg +/−3.0; p<0.0000l) littermates at 4 months of age (FIG. 11E).

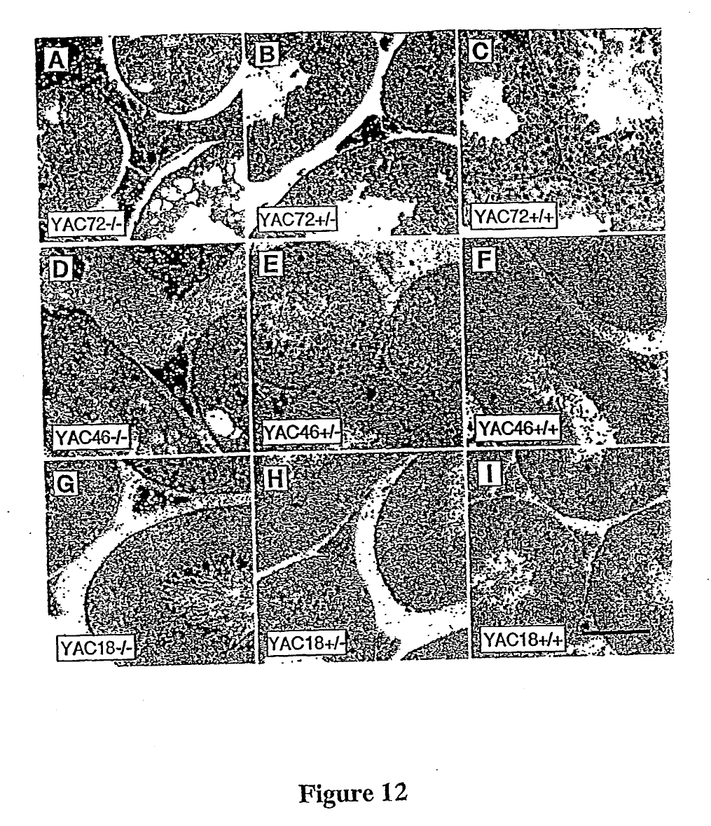

Histological examination of sections from the testes of adult mice expressing expanded mutant htt (YAC46−/− or YAC72−/−) stained with toluidine blue revealed massive disruption of spermatogenesis in the seminiferous tubules. The seminiferous tubules from these mice were full of large vacuoles and dying cells (FIGS. 12A and D), but the spermatagonial stem cells and Sertoli cells close to the basement lamina appeared relatively normal in appearance and number. Depletion, but not absence, of cells at later stages of spermatogenesis (spermatocytes and spermatids) was evident in all layers of degenerating tubules. Degenerating cells at various stages of development were identified, suggesting that the spermatogenic defect caused by mutant htt is not limited to a single stage of development or due to defective maturation of spermatocytes. The normal stratified organization of cells within these seminiferous tubules was completely disrupted. Rarely, late spermatids were identified in the outer cell layer, but mature spermatazoa were not found in the lumen of these tubules, which were often filled with cellular debris (FIGS. 12A and 12D). Leydig cells appeared to be unaffected in the stromal interstitial tissue between degenerating tubules.

Cellular Degeneration in Mice Expressing Mutant Htt Can Be Blocked By Expression of Endogenous htt and is CAG-Length Dependent

This testicular degeneration was most striking in the testes of YAC72−/− mice (FIG. 12A). Occasionally vacuolisation and cellular degeneration was seen in the testes from YAC72+/− mice expressing 50% of endogenous htt levels (FIG. 12B), but these mice were able to produce mature sperm (FIG. 11C). Increasing endogenous htt expression to 100% of normal levels in YAC72 mice completely rescued the degenerative testicular phenotype (FIG. 12C). Normal stratified organization was restored to the seminiferous tubules, and no vacuolization or increased numbers of degenerating cells was present. Mature spermatozoa were found in the lumen of seminiferous tubules from YAC72+/+ mice (FIG. 12C, arrow) and these mice had normal fertility. These results suggest that the testicular cell death caused by the expression of polyglutamine expanded htt in transgenic mice could be completely blocked by increasing the level of endogenous htt.

The testicular cell death caused by the expression of polyglutamine expanded htt in transgenic mice (YAC46−/− and YAC72−/−) mice does not occur in mice expressing the same human transgene without the CAG repeat expansion (YAC18−/− mice, FIG. 12G). YAC18 mice lacking endogenous htt had normal morphology of the seminiferous tubules, no evidence of increased testicular cell death, and normal fertility. No significant effect was seen on YAC18 mice when levels of endogenous htt were increased to 50 or 100% of the normal htt levels (FIGS. 12H and 12I).

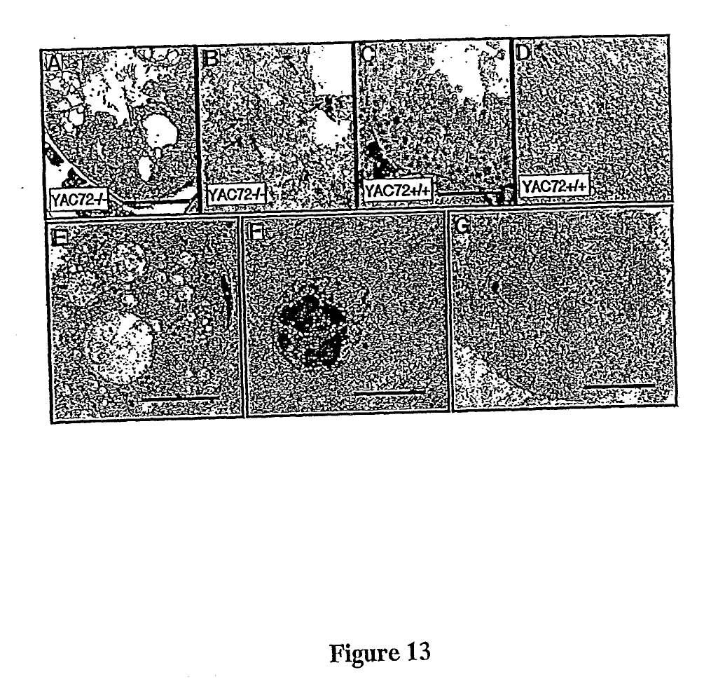

Histological examination of toluidine blue stained sections from the testes of adult mice expressing mutant htt revealed massive cellular death in multiple layers of the seminiferous tubules (FIG. 13A). TUNEL™ labelling (FIG. 13B, arrows) confirmed apoptotic nature of the widespread cell death in the testes of YAC72−/−. Despite the drastically reduced numbers of cells within seminiferous tubules from the YAC72−/− testes, the average number of TUNEL™ positive cells (3.2 per 20× field) was ˜10-fold higher in these sections than in sections from YAC72+/+ testes containing normal numbers of spermatogenic cells (0.3 per 20× field). No increased testicular cell death was observed in YAC72 mice expressing 100% of normal levels of endogenous htt (YAC72+/+) either by toluidine blue (FIG. 13C) or by TUNEL™ labeling (FIG. 13D).

Ultrastructural Analysis of the testes in YAC72 mice nullizygous for endogenous Htt (−/−) revealed large numbers of degenerating spermatids with diffuse cytoplasmic vacuolisation (FIG. 13E). Shrunken degenerating spermatids, with condensed nuclei and electron dense cytoplasm, were phagocytosed and degraded by Sertoli cells (FIG. 13F), suggestive of ongoing apoptosis and confirming the TUNEL™ findings. Multinucleated giant cells were found throughout the testes of YAC72−/− mice. These cells result from the opening of intercellular bridges between clones of spermatogenic cells. Importantly, no such degenerative phenotype was found by ultrastructural analysis of the testes of YAC18 mice.

Abnormal Protein Aggregates Occur in the Testes of Mice Expressing Mutant Htt

Ultrastructural analysis of the testes of YAC72−/− mice revealed the presence of occasional abnormal aggregates of intracellular protein (arrows) within spermatids (FIG. 14A), Sertoli cells (FIG. 14B), and sperm tails (FIG. 14C). These aggregates were rare, and found at an incidence of much less than one per high-powered field. No protein aggregates were identified in in YAC18−/− mice. Ectopic microtubule bundles (FIG. 14D) and manchettes (FIG. 14E) were also occasionally identified (arrows) within spermatogonia and spermatids respectively. Also interesting is the observation that actin-containing adhesion plaques (ectoplasmic specializations) that occur in Sertoli cell cortical cytoplasm in regions of adhesion to spermatids often occurred in ectopic positions. Normally these structures occur in regions of attachment only to spermatid heads. In YAC72−/− Sertoli cells, ectoplasmic specializations were observed to completely surround elongate spermatids that had re-acquired a circular form.

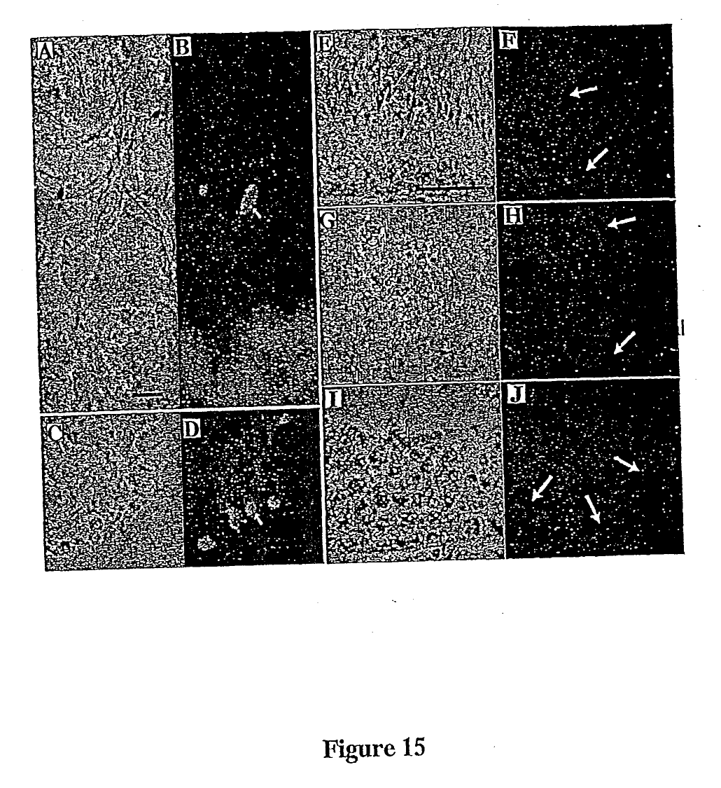

Immunocytochemical analysis of htt localization in the testes of YAC72−/− mice revealed that protein aggregates within a small numbers of degenerating spermatids (FIGS. 15A and 15B) contain htt. Similar htt immunoreactivity was identified in aggregates within Sertoli cells adjacent to the basal lamina of degenerating seminiferous tubules (FIGS. 15C and 15D). Labelling of filamentous actin with fluorescent phallotoxin revealed altered localization of filaments within the testes of YAC72−/− mice (FIGS. 15I and 15J). In YAC72+/+ (FIGS. 15E and 15F), actin filaments were concentrated in Sertoli cell adhesion plaques (ectoplasmic specializations) found apically in association with spermatid heads and basally in association with junction complexes between neighboring Sertoli cells. In YAC72−/− tissue (FIGS. 15I and 15J) actin filaments occur in linear arrays perpendicular to the tubule wall and in areas not directly related to spermatid heads (arrows with asterisks in FIG. 15J).

Thus, wild-type htt can significantly reduce the cellular toxicity of mutant htt in vivo. Expression of human htt with an expanded polyglutamine tract (46 and 72 polyglutamines) in the absence of wild-type htt results in male infertility, and massive apoptotic cell death in the testes in all phases of spermatogenesis. The cell death can be modulated by the expression of normal htt. For example, mice expressing human htt with 46 or 72 polyglutamines have no evidence for testicular atrophy or apoptosis in the testes when wild-type htt is expressed from both Hdh alleles in the mouse. An intermediate phenotype is seen in these mice (YAC46 and 72) on the background of heterozygosity for targeted disruption in the mouse Hdh gene (YAC46 +/− or YAC72+/−). The severity of the testicular atrophy and apoptotic cell death is also modulated by the length of the polyglutamine repeat. YAC72 transgenic mice require higher levels of wild-type htt (+/+) than YAC46 transgenic mice (+/−) to prevent testicular degeneration. Abnormal protein aggregates, that contain htt, are occasionally found both in Sertoli cells and in spermatogenic cells in the testis of YAC72−/− mice. Also, structures containing cytoskeletal elements form ectopically in these mice suggesting that alterations in either the targeting of cytoskeletal elements to specific positions in the cells or of cytoskeletal function may be a mechanism promoting the massive apoptosis observed. Thus, disruption of normal cytoskeletal organization may play a role in mediating the toxic effect of mutant htt.

One of the normal functions of htt in the brain may be to protect cells against pro-apoptotic stimuli, and partial loss of this function may underlie some of the selective vulnerability of striatal neurons to cell death in HD.

A number of observations indicate that the ultimate outcome of htt toxicity in the testis is the apoptotic loss of spermatogenic cells. The dramatic vacuolisation within the seminiferous tubules, TUNEL™ staining of spermatogenic cells, and the ectopic positioning of manchettes (microtubule structures associated with spermatid nucleii) in these cells are consistent with this conclusion. The presence of giant cells in the epithelium, the occurrence of large phagosomes containing spermatids in Sertoli cells and the obviously reduced numbers of spermatogenic cells in the epithelium demonstrated by our electron microscopy analysis all point to the conclusion that the primary testicular phenotype in YAC72−/− mice is apoptotic death of spermatogenic cells, particularly of spermatids.

Although morphological changes and cell loss are most dramatic in the spermatogenic cell population in YAC72−/− testes, Sertoli cells also express abnormal features. Two of these features are the presence of protein aggregates in the cytoplasm and the ectopic positioning of actin filament containing junction plaques normally found in regions adjacent to spermatid heads. Abnormal positioning of ectoplasmic specializations adjacent to spermatids cells may be a response to a primary defect in the associated spermatogenic cells. The presence of normally positioned junction plaques in basal regions of attachment to neighbouring Sertoli cells is consistent with this conclusion. These findings suggest that mutant htt causes an intrinsic polyglutamine-mediated apoptotic cell death within spermatogenic cells, which can be blocked by wild-type htt.

The data provided herein demonstrate strong in vivo evidence that wild-type htt can significantly modulate the apoptotic toxicity of mutant htt, and we suggest that wild-type htt may normally have an anti-apoptotic function.

Role of Wild-Type Htt on Cell Proliferation and Tumor Formation In Vivo

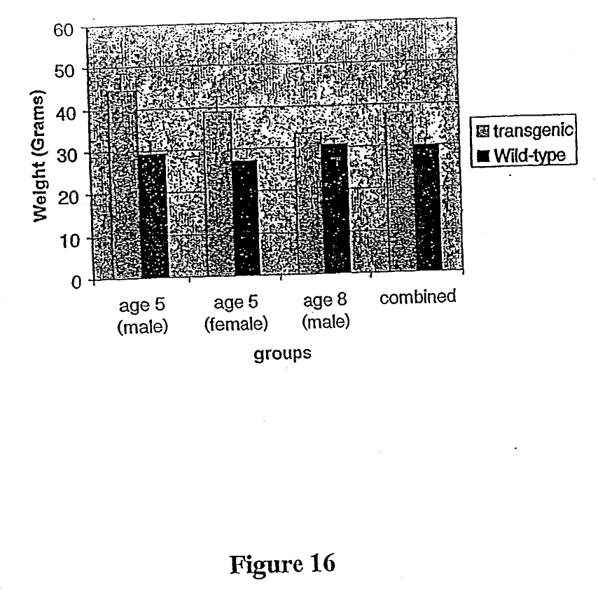

One of the roles of the pro-survival (e.g. anti-apoptotic) function of wild-type htt may be in control of tissue mass and cellular proliferation. Increased levels of wild-type htt expression can lead to increased body tissue mass and may predispose mice to the development of tumors. The effect of over-expression of human wild-type htt on the body weight of transgenic mice was compared to wild-type mice (FIG. 16). Transgenic mice aged 5-8 months old that over-express wild-type htt (n=11) had a significantly increased total body mass compared to sex and age matched control mice (n=12). The average body weight was approximately 25% greater for the transgenic mice compared to wild-type. The increased body weight of the transgenic mice suggests that the wild-type htt is causing increased cellular survival or proliferation in general in these mice, and that increasing wild-type htt function increases the number of cells in the body. Over production of cells in the body can have a variety of unwanted outcomes including but not limited to obesity or cancer. This may be due to decreased naturally occurring apoptosis or increased cellular proliferation.

A retrospective analysis was performed to examine the total numbers of mice (identified by the animal colony health staff) that developed tumors in the colony of transgenic mice. The records of all mice diagnosed with tumors over a 6 month period were examined, in a group of mice over-expressing wild-type human htt and their wild-type littermates. The vast majority of mice that developed tumors were found to be transgenic mice that over-expressed wild-type htt (38/45 or 84.5% of mice identified with tumors were transgenic). The average age of tumor development was younger in the transgenic group 13.8 months compared to 17.1 months for wild-type littermates. The majority of identified tumors in male mice were testicular tumors. Htt is expressed at very high levels in the testes suggesting that this tissue is at increased risk of tumor development when htt levels are increased.

Role of Htt on Cell Proliferation In Vitro

Embryonic stem cells that had decreased levels or complete absence of htt were exposed to hematopoetic cytokines that stimulate cellular proliferation and found to have less response (less proliferation) than stem cells that had the normal levels of htt. This indicates that htt can have both anti-apoptotic and proliferative functions.

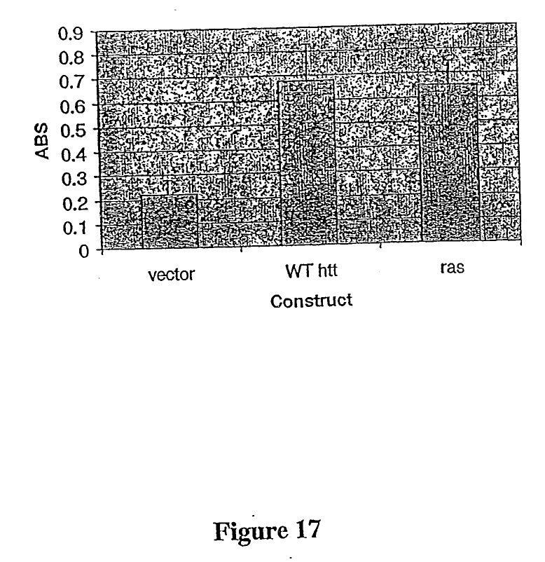

The ability of NIH3T3 cells to overcome contact inhibition when transfected with different gene constructs has been used for many years to assess oncogenic potential of certain genes and to identify potential oncogenes. Cellular proliferation was assessed following transient transfections of NIH3T3 cells using a standardised colorometric assay of cell number. The effect of htt on cellular growth was examined and compared to the effect of a known oncogene, ras (FIG. 17). NIH3T3 cells transfected with htt constructs show a significant increase in cellular proliferation (5-6 fold increase in proliferation compared to control vector) that is similar to the effect of ras.

The in vitro and in vivo data demonstrate that the anti-apoptotic function of htt plays a role in controlling cellular proliferation and that alterations in this function can cause cancer or neurodegeneration depending on whether htt function is increased or decreased.

Phosphorylation of Htt and Its Role in Cellular Proliferation

The amino acid sequence of htt contains several sequences identified as being consensus sequences for phosphorylation by the protein kinase AKT, which is known to have pro-proliferative and anti-cell death functions, and is involved in signaling pathways known to be involved in causing cancer. AKT is a Serine (S)/Threonine (T) kinase which preferentially phosphorylates S/T residues lying in RXRXXS/T sequences, where X is any amino acid, and S/T means either S or T. This, or a closely related, motif may be found in human htt at amino acid positions:

S421 (serine at amino acid position 421) Sequence=GGRSRSGSIVELIAG,

T2068 (threonine at amino acid position 2068) Sequence=LDRFRLSTMQDSLSP,

T1024 (threonine at amino acid position 1024) Sequence=TSTTRALTFGCCEAL,

where residues predicted to be phosphorylated are indicated in bold font.

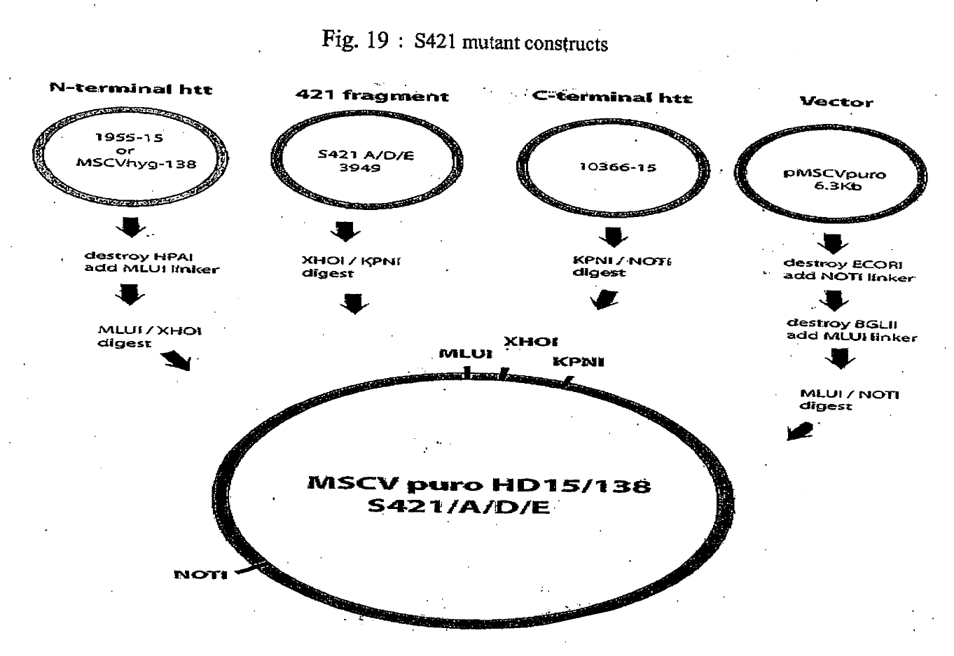

S421 mutants of full-length htt (unphosphorylatable and constitutively phosphorylated at serine 421) were constructed (FIG. 19). PCR mutagenesis was performed to replace serine 421 with alanine (S421A, which is unphosphorylatable) and aspartate or glutamate (S421D or S421E, which mimic constitutive phosphorylation), followed by cloning into 3949 construct containing truncated huntingtin protein. The resulting constructs were then cloned into full length htt and into the commercially available MSCV murine retroviral vector. Sequence analysis conducted across the mutated region and across the polyglutamine region of the constructs, as well as transient transfections of the constructs into 293T cells, which resulted in proteins of the expected mass, confirmed the integrity of the constructs.

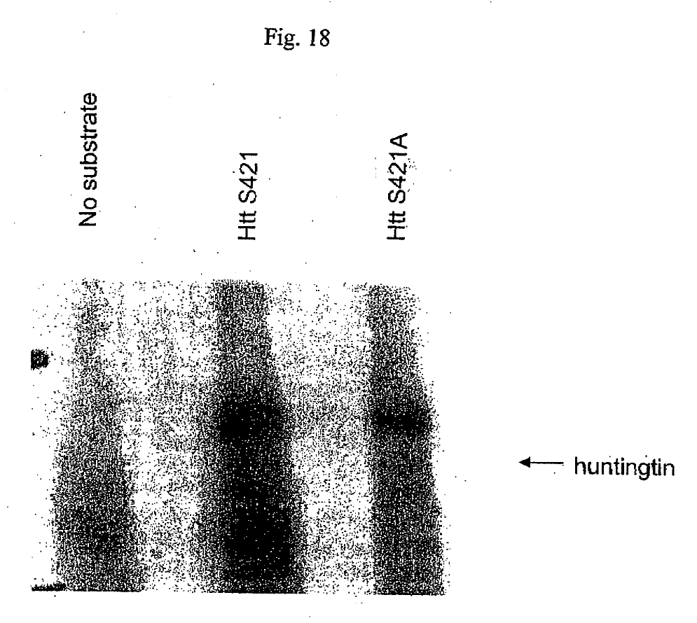

Serine 421 of huntingtin can be phosphorylated by purified recombinant active AKT in vitro (FIG. 18). Immunoprecipitated recombinant protein containing the N-terminal one-third of htt was used as a substrate for AKT. Phosphorylation was detected by autoradiography since radiolabelled ATP was a component of the in vitro kinase reaction. When serine 421 was mutated to alanine (S421A), a non-phosphorylatable amino acid, 64% of the phosphorylation signal on huntingtin was ablated. These results demonstrate that AKT can phosphorylate htt in vitro and that this reaction is specific for serine 421. Other sites for AKT phosphorylation are threonines 1024 and 2068.

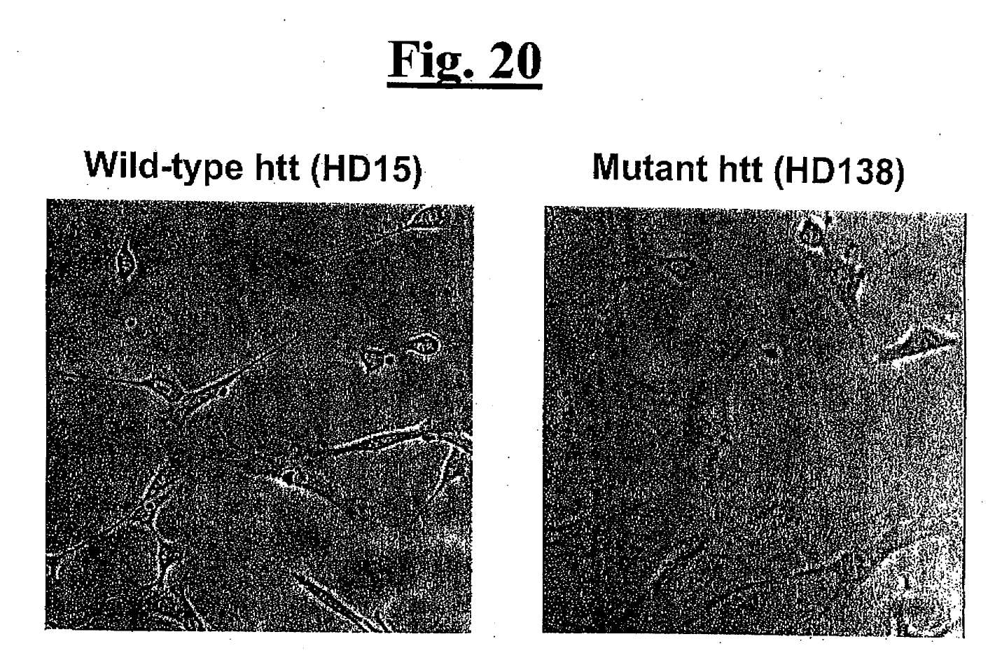

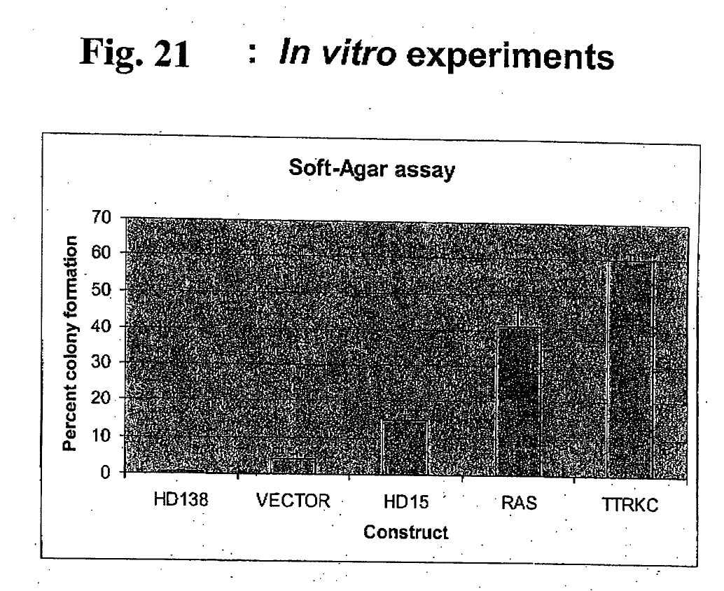

Retroviral introduction of the S421 mutants into NIH3T3 cells was used to examine the oncogenic (proliferative) effects of phosphorylation on serine 421. Stable retroviral transfection of NIH3T3 cells with wild-type huntingtin leads to malignant, morphologic transformation (FIG. 20). To test the oncogenic potential of htt and to examine the effect of phosphorylation of htt at S421 on cell proliferation, a soft agar assay was conducted in NIH3T3 cells (FIG. 21). Comparison of the colony formation capabilities of NIH3T3 cells transfected with wild-type htt, control vector, mutant htt, or oncogene (e.g., RAS) indicated that wild-type htt expression has a 3 fold increase in number of colonies (more colonies=more like cancer cells) over control cells (about 15% compared to about 5%), but less than a dramatic oncogene like RAS (which leads to about 40% colonies). Mutant htt (HD138) transfection leads to no (0%) soft agar colonies.

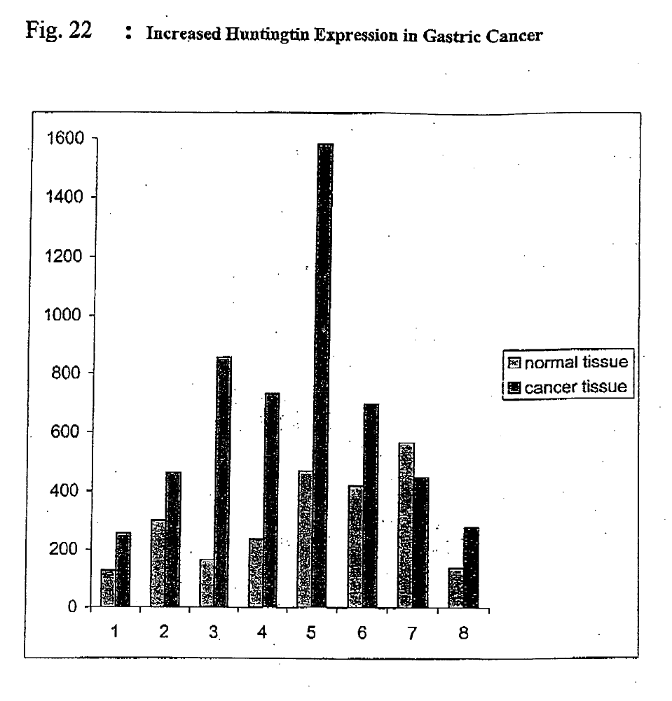

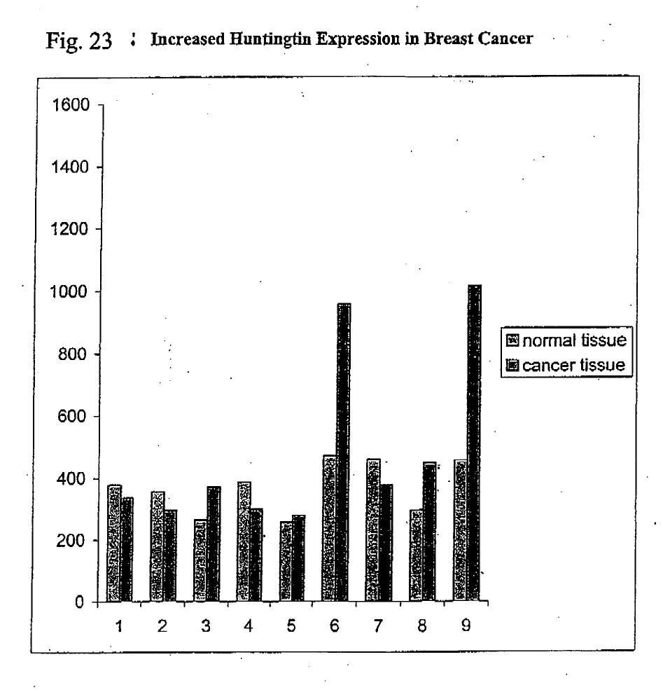

Screening human tumors for increased htt expression levels, either through screening human lymphoma samples, screening NCI 60 cell lysates, or screening cancer tissue arrays, indicated that increased levels of htt are found in some forms of human tumors such as gastric and breast tumors (FIGS. 22 and 23). Accordingly, measurement of htt levels may be used as a diagnostic for cancer or cell proliferation, where an increased level of htt, compared to normal tissue, is an indication of aberrant cell proliferation or cancer.

Htt Mediates Anoikis Resistance

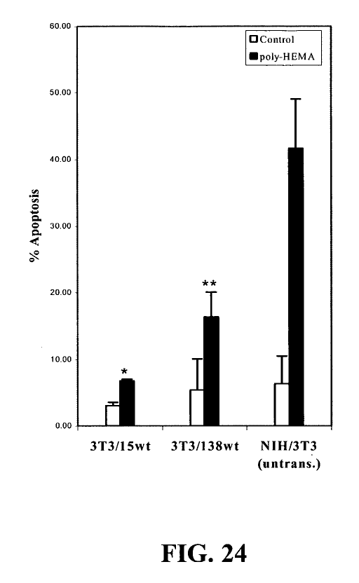

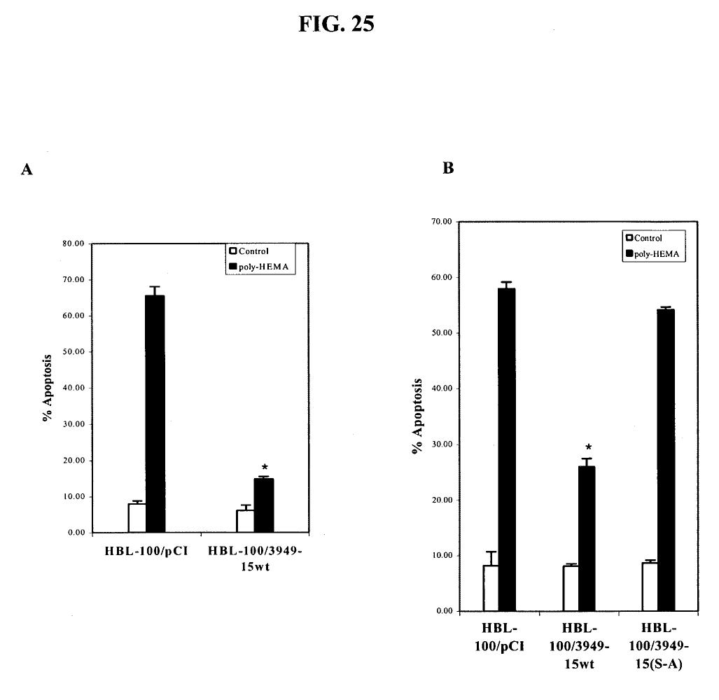

Using the murine transformed fibroblast cell line, NIH/3T3, the anoikis-resistance properties of wild type htt (15 glutamine repeat) and mutant htt (138 glutamine repeat) were investigated. NIH/3T3 cells require a matrix for attachment in order to experience optimum growing conditions. When detached from the supporting matrix, they experience a high rate of apoptosis. NIH/3T3 cells were transfected with a mammalian expression vector containing wild type htt (15 glutamine repeat) or mutant htt (138 glutamine repeat). Untransfected and transfected NIH/3T3 cells were grown on cell culture plates, whose plastic surface served as a substrate for matrix deposition and subsequent cellular attachment, or on poly-HEMA coated cell culture plates. The nonionic nature of poly-HEMA inhibits both matrix deposition and subsequent cell attachment. A poly-HEMA concentration of 10 mg/ml (1 mg/cm²) was used and has been shown to be adequate in preventing cell adhesion to tissue culture plastic. The cells were cultured in DMEM with 10% serum for 8 hours under attached conditions on plastic plates or under detached conditions on poly-HEMA-coated plastic plates. Cells were harvested, counted and viability was assessed via annexin-V-FITC and propidium iodide (PI) staining. Protection from cell death induced by anoikis is shown in 3T3/15wt and 3T3/138wt (*p<0.05, **p<0.05 with respect to parental cells) (FIG. 24).

All cells grew well on normal cell culture plates, as evident by a low rate of apoptosis, but both wild-type and mutant htt-expressing NIH/3T3 cells grown in poly-HEMA coated plates also grew well, indicating a significant measure of anoikis-resistance. Untransfected NIH/3T3 cells experienced high rates of apoptosis (˜45%) when grown in poly-HEMA coated plates, as detachment resulted in anoikis directed apoptosis.

Phosphorylation of Htt Mediates Anoikis Resistance

The human breast cell line HBL-100, transformed by the SV40 virus large T antigen with epithelial like morphology, was used to investigate the importance of the huntingtin serine residue at position 421 with respect to phosphorylation towards anoikis resistant, anchorage independent cell growth. HBL-100 cells were transfected with a 3949-15wt huntingtin vector construct, or a similar vector in which the normal serine at position 421 was mutated to an alanine residue. This mutation prevents phosphorylation from occurring at that site, which is presumed to be mediated by the kinase AKT/PKB. Cells were cultured in DMEM with 5% serum for 24 hours either on untreated tissue culture plastic or on poly-HEMA-coated plastic plates. Cells were harvested, counted and viability was assessed via annexin-V-FITC and propidium iodide (PI) staining.

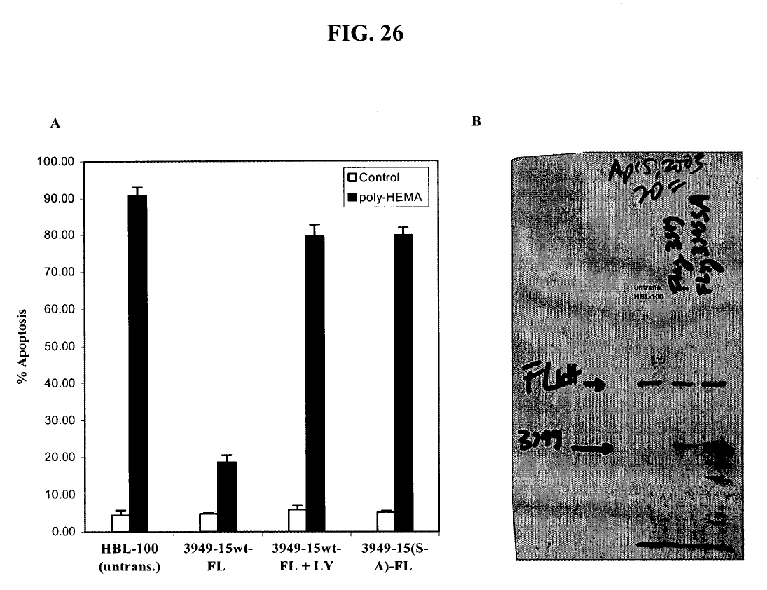

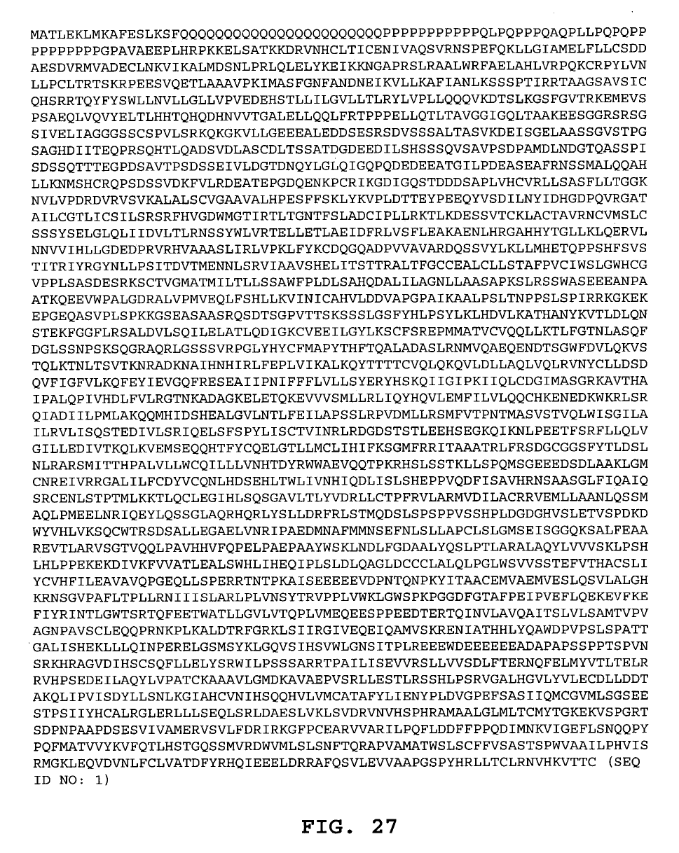

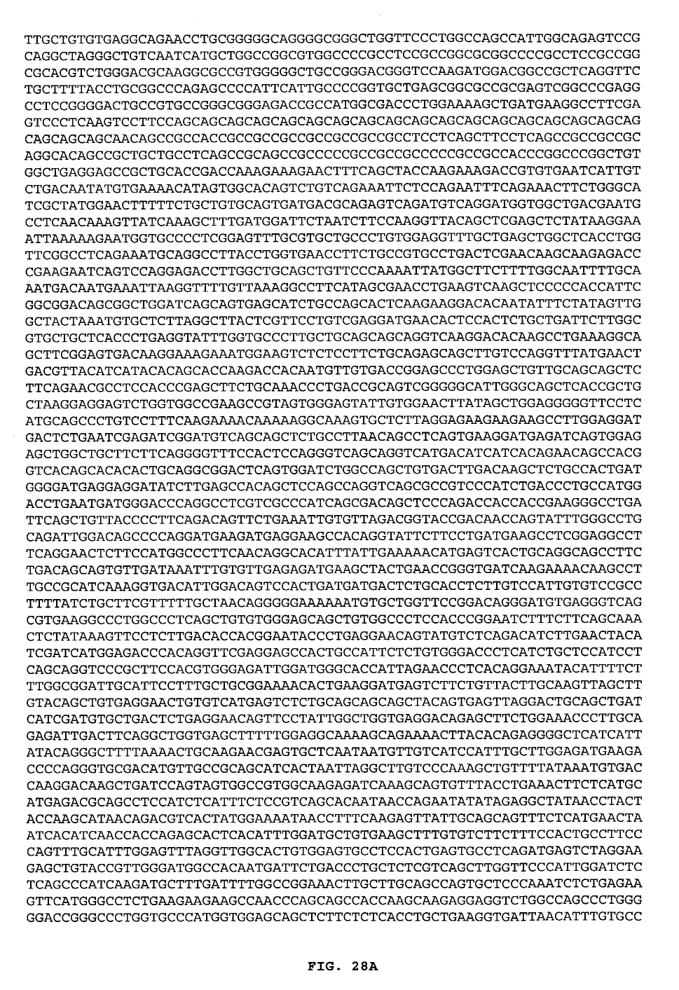

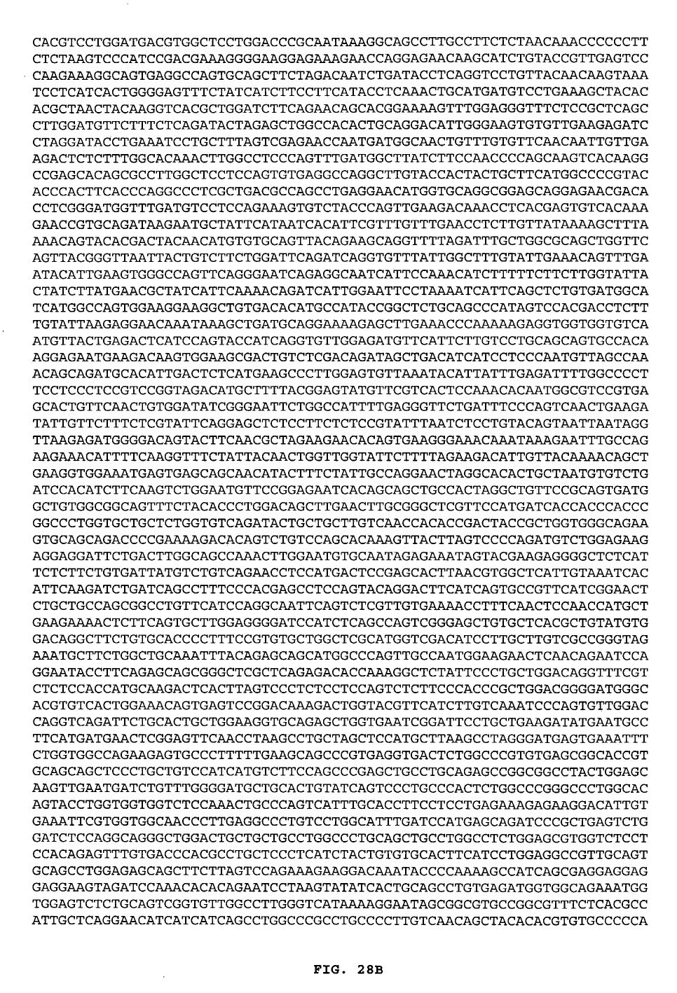

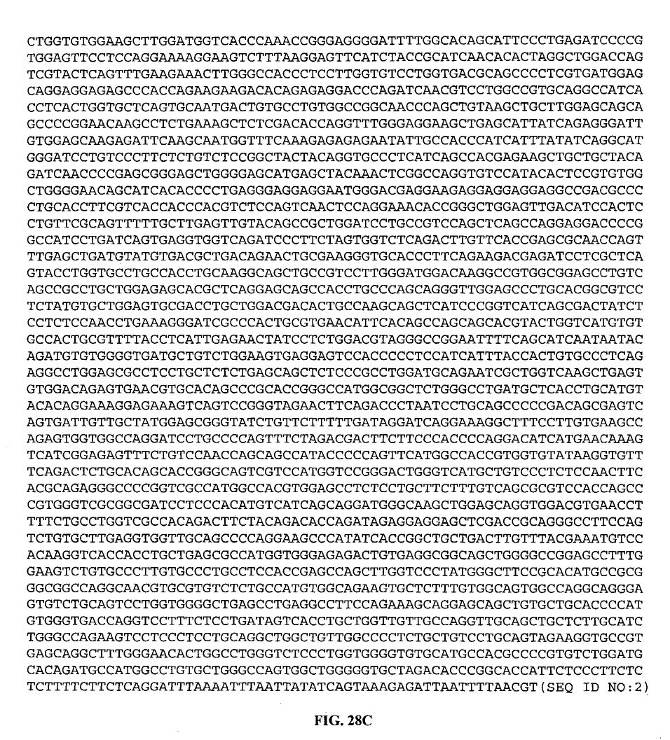

Once again, normal HBL-100 cells grew well in normal cell culture plates where attachment was facilitated, but experienced a high degree of anoikis mediated apoptosis when grown on poly-HEMA coated plates where attachment was inhibited (FIGS. 26A-B). HBL-100 cells transfected with the normal huntingtin construct displayed pro-survival, anti-apoptotic properties as they grew well whether attached to a matrix or in a detached state. The pro-survival effect of huntingtin expression was negated when the serine at position 421 was mutated to an alanine residue (FIG. 26B). HBL-100 cells expressing the 3949-15(S-A) construct behaved similarly to untransfected cells, indicating that the anoikis-resistant, anti-apoptotic signal due to huntingtin was mediated by phosphorylation of S421 of the huntingtin protein, presumably by AKT/PKB.

Kinase Inhibition Studies For Anoikis Mediated Signal Transduction.

HBL-100 cells were transiently transfected with a FLAG-tagged 3949-15wt htt, or a FLAG-tagged (S-A) unphosphorylatable mutant htt. Wild-type cells were also treated with LY294002, a specific PI-3 kinase inhibitor, at a concentration of 10 μM, to determine whether inhibition of kinase function could abrogate the induction of anoikis. Western blot analysis confirming the expression of both full length and 3949 huntingtin in the FLAG-tagged constructs (FIG. 26B). The presence of endogenous huntingtin in the parental cells is also shown. The pro-survival effect of huntingtin is observed in the wild-type (*p<0.0001 with respect to parental, LY-treated, and mutant cells) (FIG. 26A).

Untransfected HBL-100 cells and the S421A mutant grew well when allowed to attach to cell culture plates, however both grew poorly in a detached state. By comparison, the normal huntingtin construct grew well irrespective of attachment. These findings underline the importance of phosphorylation, for example, at position 421 of the huntingtin protein in order for that protein to mediate the anti-apoptotic, anoikis resistant signal transduction. These earlier fmdings were again supported by the current experiments in which a specific phophoinositol-3 (PI-3) kinase inhibitor, LY294002 (LY), was used to block phosphorylation of the huntingtin serine residue at position 421. Blocking of PI-3 kinase by LY prevents activation of AKT/PKB, thus preventing the phosphorylation of serine 421 of huntingtin. Indeed LY prevented anoikis resistant cell growth of detached HBL-100 cells in a manner nearly identical to the huntingtin S421A construct. These findings support the theory that AKT/PKB mediated phosphorylation of huntingtin at the serine residue positioned at 421 is instrumental in allowing cells to grow independent of cell attachment, and therefore subvert the anoikis signal transduction pathway that promotes apoptosis.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains, and may be applied to the essential features set forth herein and in the scope of the appended claims.

All patents, patent applications, and publications referred to herein are hereby incorporated by reference in their entirety to the same extent as if each individual patent, patent application, or publication was specifically and individually indicated to be incorporated by reference in its entirety.

What is claimed is:

1. A method of modulating cell survival in a subject in need thereof, said method comprising administering an effective amount of a huntingtin protein or a biologically-active fragment or variant thereof.

2. A method of treatment or prophylaxis of a cell degenerative disease, said method comprising administering a huntingtin protein or a biologically-active fragment or variant thereof, to a subject in need thereof.

3. A method of treatment or prophylaxis of a cell proliferation disease, said method comprising administering a huntingtin protein or a biologically-active fragment or variant thereof, to a subject in need thereof.

4. The method of any one of claims 1 through 3, wherein said variant is selected from the group consisting of an agonist of a huntingtin protein, and an antagonist of a huntingtin protein.

5. The method of claim 1, wherein said modulating comprises modulating cell death or apoptosis.

6. The method of claim 1, wherein said modulating comprises modulating cell proliferation.

7. The method of any one of claims 1 through 3, wherein said huntingtin protein is selected from the group consisting of full-length huntingtin protein, wild-type huntingtin protein, and mutant huntingtin protein.

8. The method of any one of claims 1 through 3, wherein said huntingtin protein is selected from the group consisting of phosphorylated huntingtin protein, unphosphorylated huntingtin protein, and constitutively phosphorylated huntingtin protein.

9. The method of any one of claims 1 through 3, wherein said biologically-active fragment is a C-terminal fragment of a huntingtin protein.

10. The method of claim 9, wherein said C-terminal fragment consists essentially of amino acids 585-3144 of the human huntingtin protein (SEQ ID NO: 1).

11. The method of claim 9, wherein said C-terminal fragment consists essentially of about the C-terminal half of the human huntingtin protein (SEQ ID NO: 1).

12. The method of claim 11, wherein said phosphorylated huntingtin protein is phosphorylated on serine 421 of the human huntingtin protein (SEQ ID NO: 1).

13. The method of claim 11, wherein said phosphorylated huntingtin protein is phosphorylated on threonine 1024 or threonine 2068 of the human huntingtin protein (SEQ ID NO: 1).

14. The method of any one of claims 1 through 3, comprising administering a nucleic acid molecule encoding said huntingtin protein or biologically-active fragment thereof.

15. The method of any one of claims 1 through 3, comprising administering an antibody that specifically binds said huntingtin protein or biologically-active fragment thereof.

16. The method of any one of claims 1 through 3, comprising administering a nucleic acid molecule complementary to a nucleic acid encoding said huntingtin protein or biologically-active fragment thereof.

17. The method of any one of claims 1 through 3, wherein said subject is human.

18. The method of claim 2, wherein said cell degenerative disease is selected from the group consisting of apoptotic diseases, neurodegenerative diseases, Huntington's disease, Alzheimer's disease, Parkinson's disease, amyotrophic lateral sclerosis, multiple sclerosis, polyglutamine diseases, spinocerebellar ataxias, autosomal dominant cerebellar ataxia with retinal degeneration, spinobulbar muscular atrophy (SBMA), dentatorubralpallidoluysian atrophy (DRPLA), Machado-Joseph disease, stroke, epilepsy, spinal cord injury, physical trauma, and retinal degeneration.

19. The method of claim 3, wherein said cell proliferative disease is selected from the group consisting of cancer, testicular cancers, embryonic cancers, leukemias, haematopoietic diseases, psoriasis, atherosclerosis, inflammatory diseases, and dermatological diseases.

20. A method of assaying a test compound, said method comprising:
(a) providing a system comprising a huntingtin protein or biologically-active fragment thereof,
(b) contacting said system with said test compound, and
(c) determining whether said test compound modulates the phosphorylation of said huntingtin protein or fragment thereof.

21. The method of claim 20, wherein said assaying is done in vitro and further comprises providing a kinase capable of phosphorylating said huntingtin protein or biologically-active fragment thereof.

22. The method of claim 21, wherein said kinase is AKT.

23. A method of assaying a test compound, said method comprising:
(a) providing a system comprising a huntingtin protein or biologically-active fragment thereof,
(b) contacting said system with said test compound, and
(c) determining whether said test compound modulates a function of said huntingtin protein or fragment thereof, wherein said function is selected from the group consisting of apoptosis inhibition, aggregation inhibition, and cell proliferation.

24. The method of claim 20 or 23, wherein said variant is selected from the group consisting of an agonist of a huntingtin protein, and an antagonist of a huntingtin protein.

25. The method of claim 20 or 23, wherein said huntingtin protein is selected from the group consisting of full length huntingtin protein, wild-type huntingtin protein, and mutant huntingtin protein.

26. The method of claim 23, wherein said huntingtin protein is selected from the group consisting of phosphorylated huntingtin protein, unphosphorylated huntingtin protein, and constitutively phosphorylated huntingtin protein.

27. The method of claim 20 or 23, wherein said biologically-active fragment is a C-terminal fragment of a huntingtin protein.

28. The method of claim 27, wherein said C-terminal fragment consists essentially of amino acids 585-3144 of the human huntingtin protein (SEQ ID NO: 1).

29. The method of claim 27, wherein said C-terminal fragment consists essentially of about the C-terminal half of the human huntingtin protein (SEQ ID NO: 1).

30. The method of claim 26, wherein said phosphorylated huntingtin protein is phosphorylated on serine 421 of the human huntingtin protein (SEQ ID NO: 1).

31. The method of claim 26, wherein said phosphorylated huntingtin protein is phosphorylated on threonine 1024 or threonine 2068 of the human huntingtin protein (SEQ ID NO: 1).

32. A method of ameliorating the cytotoxic effects of a therapeutic compound, said method comprising administering a huntingtin protein or a biologically-active fragment or variant thereof with said therapeutic compound.

33. The method of claim 32, wherein said therapeutic compound is a pro-apoptotic compound.

34. The method of claim 33, wherein said pro-apoptotic compound is tamoxifen.

35. A method for diagnosing cell proliferation, said method comprising determining the level of a huntingtin protein or nucleic acid molecule in a test sample and in a control sample, wherein said test sample is positive for cell proliferation if said level is higher in said test sample than in said control sample.

36. The method of claim 35, wherein said test sample is selected from the group consisting of a gastric cancer sample and a breast cancer sample.


SEQUENCE LISTING (6 sequences)

SEQ ID NO: 1 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 2 - DNA, 10348 bp, Homo sapiens
ttgctgtgtgaggcagaacctgcgggggcaggggcgggctggttccctggccagccattggcagagtccgcaggctagggctgtcaatcatgctggccggcgtggccccgcctccgccggcgcggccccgcctccgccggcgcacgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctcaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgtgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgt

SEQ ID NO: 3 - protein, 6 aa, Artificial
  Sequence Motif
RXRXXX

SEQ ID NO: 4 - protein, 15 aa, Homo sapiens
GGRSRSGSIVELIAG

SEQ ID NO: 5 - protein, 15 aa, Homo sapiens
LDRFRLSTMQDSLSP

SEQ ID NO: 6 - protein, 15 aa, Homo sapiens
TSTTRALTFGCCEAL